(19) United States
(12) Patent Application Publication
(10) Pub. No.: US 2024/0307474 A1
(43) Pub. Date: Sep. 19, 2024

(54) Fermented Uni-sourced Nanoemulsion of Nigella sativa or Cannabis sativa For Use in Medical, Cosmetic, and Recreational Indications with a Method of Its Production and Use

(71) Applicant: APEX MEDICAL DEVICE DESIGN, LLC, Dublin, OH (US)

(72) Inventors: Nedaa Abdulghani Nasif Al Jasim, Dublin, OH (US); Noor Z. Ahmed, Dublin, OH (US)

(73) Assignee: APEX MEDICAL DEVICE DESIGN, LLC, Dublin, OH (US)

(21) Appl. No.: 18/577,643

(22) PCT Filed: Jul. 6, 2022
(86) PCT No.: PCT/US2022/036272

§ 371 Date: Jan. 8, 2024

Related U.S. Application Data
(60) Provisional application No. 63/218,898, filed on Jul. 6, 2021.

(51) Int. Cl. A61K 36/71; A61K 8/35; A61K 8/60; A61K 8/9789; A61K 8/9794; A61K 9/107; A61K 31/00; A61K 31/122; A61K 31/575; A61K 31/704; A61K 36/9066; A61K 47/24; A61Q 19/08

(52) U.S. Cl. CPC A61K 36/71; A61K 8/355; A61K 8/604; A61K 8/9789; A61K 8/9794; A61K 9/1075; A61K 31/122; A61K 31/575; A61K 31/658; A61K 31/704; A61K 36/3482; A61K 36/9066; A61K 47/24; A61Q 19/08

(57) ABSTRACT

A botanical uni-sourced drug-loaded lipid-based nanoemulsion produced from one single medicinal herb such as Nigella sativa, Cannabis sativa, or Curcuma longa, wherein the herb acts as a comprehensive provider for the bioactive phytochemicals, oil phase, saponin surfactant, co-surfactant, proteins, and carbohydrates to produce the uni-sourced safe, clear, stable lipid-based drug delivery system. The nanoemulsion can be formulated into oral dosage form including nanoemulsion, microemulsion and liposomes, wherein the extraction and emulsification are carried out by the addition of water-soluble organic solvent, wherein the product can be further fermented and esterified for improved stability, wherein Nigella sativa uni-sourced nanocompositions are used as first-in-class oral formulation to relax coily hair targeting the arrector pili muscle in the hair root, anticancer, anti-tuberculosis, anti-COVID-19; and antiviral treatment, Cannabis nanocompositions are used for medical or recreational purposes, and Curcuma longa is used for nutraceutical and treatment purposes.

### CROSS-REFERENCE TO RELATED APPLICATIONS

This application claims the benefit under 35 U.S.C. § 119 of U.S. Provisional Application Ser. No. 63/218,898, filed Jul. 6, 2021, which is incorporated herein by reference.

### STATEMENT REGARDING FEDERALLY SPONSORED RESEARCH

Not applicable.

### BACKGROUND OF THE INVENTION

The present disclosure generally relates to a botanical lipid-based drug-loaded nanomedicine and methods of manufacturing the nanomedicine. More specifically, the disclosure relates to a nanomedicine that acts as a hydrophobic drug delivery system used in the formulation of oral dosage forms and for local application.

Regulatory aspects of Lipid-based Drug Delivery System (LBDDs)

A LBDDs is a multi-component nanocarrier loaded with active pharmaceutical ingredients. According to the Food and Drug Administration, there are two points to consider when considering if a product involves the application of nanotechnology: first, whether the material or end products is engineered to have certain dimensions in the nanoscale range approximately 1-100 nm, or up to one micrometer (1,000 nm). Second, is whether a material or end product exhibit certain properties or phenomena, including physical or chemical properties or biological effects, that are attributed to its dimensions (Guidance for the industry: considering whether an FDA-regulated products involves the application of nanotechnology, June 2014).

The product properties that are attributed to the dimensions in the process of nano-conversion are described by the Guidelines for Evaluation of Nanopharmaceuticals in India (October 2019) that indicates that the conversion of any material in nanoscale results in alteration of its optical, mechanical, physicochemical, biological, and other properties. These newly acquired (novel) properties of the materials due to conversion into nanoscale can be utilized to produce useful products in the pharmaceutical and cosmetic industry.

According to published research, the change in the optical properties include the formation of a transparent or translucent composition, and the color of the composition. The mechanical and physicochemical properties include the reduction of the interfacial tension between the oil (organic) phase and water phase; hence the oil becomes water miscible system, the system is stable, and the smell and the taste are improved.

According to the FDA Guidance for the industry: Liposome Drug Product, April 2018, the liposome drug product has a composition of: (1) Drug substance; (2) Lipids (naturally sourced complex lipids mixtures, synthetic, semisynthetic, and modified); (3) Nonlipid components of liposomes; (4) Nonliposome inactive ingredients (e.g., buffer components with different concentrations and PH).

The roadblock to clinical translation of nanopharmaceuticals.

Medicinal plants have been discovered and used in traditional medicine practices since prehistoric times. Numerous bioactive phytochemicals with potential or established biological activity have been identified, however, the majority of the phytochemicals are hydrophobic (repels water and dissolve in oil) and are challenging for the formulation scientists with regard to solubility and bioavailability. Further, many of the bioactive phytochemicals are unstable in aqueous solutions and during storage due to photodegradation and chemical disintegration.

To overcome the roadblock in clinical translation of these hydrophobic unstable bioactive phytochemicals (bioactives) they are formulated into a lipid-based drug Delivery system (LBDDs) including nanoemulsions, microemulsions, self-nanoemulsifying DDs, liposomes, and others. LBDDs are engineered to carry, protect, enhance bioavailability, and deliver the bioactives to the targeted tissues. LBDDs can be tailored to meet a wide range of product requirements and requires proper understanding of the physicochemical nature of the bioactive compound; thus, suitable carrier have to be thoughtfully selected for every therapeutic. Choosing the most appropriate technology to fabricate LBDDs that is best for a particular drug is not an easy task as there are many factors which influence the final choice (Chris Moreton, 2021; Josef Jampilek et al., 2019; El-Far et al., 2018; Shrestha et al., 2014).

The problem is that, after five decades of intensive research, only few nanomedicines entered the market for the treatment of cancer and inflammatory diseases (Mi-Kyung Lee, 2020). “Nano's potential benefits are frequently overstated or inferred to be very close to application when clear bottlenecks to commercial translation exist” (Bawa, 2019). nanomedicine development continues to focus on optimizing delivery platforms with a one-size-fits-all solution (Mitchell et all., 2021; Germain et al., 2020). In fact, the current mix-and-match approach to select components from whatever is known in the prior art aren't enabled to engineer a customized nanoformulation. Finding the right recipe that allows nanoparticle optimization is a major challenge; a suitable carrier has to be thoughtfully selected for every therapeutic compound.

Further, the traditional manufacturing methods are labor intensive, hard to reproduce, and difficult to scale up because the characteristics of nanoparticles are sensitive to manufacturing conditions (Tim Leaver, 2017).

Analysis of the problem root causes of the problem.

In order to identify the root causes of the above-identified bottleneck, herein a deep and thorough exploration of possible contributing factors in the current state of the art that will help solve the problem, including:

I. Heterogenous sources of the formulation components.

II. Mix-and-match process of optimization.

III. Challenges with current methods of production of LBDDs.

IV. Deep analyses of how the different parts of the whole interacts with each other to influence the overall outcome in this complex system.

I. Heterogenous components of LBDDs formulation.

Currently, LBDDs (including nanoemulsions, microemulsions, and liposomes) are made from heterogenous components, that means each component is derived from a different source, i.e., natural, synthetic, or semisynthetic (FDA Guidance to Industry, 2018; Mazonde et al, 2020; Josef Jampilek et al., 2019; Deshmukh et al., 2021). The selected components are planned to interact and assemble at the molecular level forming a stable drug carrier.

FIG. 1 is a flow chart that depicts the heterogenous nature of the composition of nanoemulsions (as a representative of LBDDs). Nanoemulsions is a dispersion of two immiscible fluids normally consisting of an oil phase (1) dispersed in an aqueous phase (2) that are mixed to form a single phase by means of an emulsifying agent, i.e., surfactant and co-surfactant (3-5).

The oil phase (1): Oil/lipid selection is generally based on the drug solubility; oil phase which have high drug loading is generally used for development on NE. The oil phase (1) consisting of either vegetable oils or lipids. The heterogenous sources of lipids used in the formulation of liposome drug products (Liposome Drug Product. FDA Guidance for Industry dated April 2018) are classified as naturally sourced complex lipids mixtures (e.g., egg and soy lecithin), synthetic lipids, or semisynthetic lipids (e.g., dipalmitoylphosphatidylcholine), modified lipids (e.g., polyethylene glycol PEG). Other lipids used in nanoparticle formulation include triglycerides, vegetable oils, mineral oils, free fatty acids, saturated fatty acids (lauric acid, myristic acid, capric acid), unsaturated fatty acids (oleic acid, linoleic acid, linolenic acid), fatty acid esters (ethyl or methyl esters of lauric, myristic and oleic acid) (Kumar et al, 2019; Kale and Deore, 2017) Numerous other lipids and oils are known in the art and in published articles.

Surfactants, cosurfactants, solvents and saponins (3-5): surfactants are surface active agents, also known as emulsifiers or excipients. These are molecules that promote nanoparticle formation and stability. The most used surfactants are phospholipids including phosphatidylcholine (egg or soybean lecithin), hydrogenated or synthetic phosphatidylcholine. Surfactants includes also Spans (fatty acid esters), Tweens (Tween 20,40,60, and 80, that are nonionic surfactant derivatives of fatty acid esters), Cremophor EL (castor oil derivative), amphiphilic proteins (why proteins and caseinate) (Kumar et al, 2019). An example is the U.S. patent application Ser. No. 17/291,545, titled “Composition comprising Nigella sativa oil and surface-active agents”. This invention provides a composition comprising Nigella sativa cold-pressed oil contains TQ together with a surface-active agent preferably selected from a group consisting of Vitamin E derivatives, D-alpha-tocopherol polyethylene glycol succinate (TPGS), lecithin and isolecithin. Numerous other surfactants are known in the art and in published articles.

Saponins have synergistic effect with surfactants. Examples are Quillaja saponin, proteins, polysaccharides, and lipoproteins (Vinarov et al., 2018).

Co-surfactants are small molecule surfactant, include ethyl alcohol, propanol, sorbitol, Ethyl Acetate, and others.

Solvents include ethyl alcohol or polyethylene glycol (PEG) and others.

Bioactive pharmaceutical ingredient (API) (6-7): are oil soluble (hydrophobic) compounds (6), that will be carried and delivered by the LBDDs. Nanoemulsions of botanical compounds, with a complex structure, might contain water soluble compounds dissolved in the water phase (7).

FIG. 2A depicts the molecular assembly and architecture of a nanoemulsion/microemulsion particle depicting a poor molecular assembly of the multiple building molecules that form the shell structure of nanoemulsion. This figure represents an oil-in-water nanoemulsion composed essentially of an oil micro-nano-droplet forming the lipid core (oil phase) (1), dispersed in the water phase (2). The oil core is surrounded by a shell composed of multiple molecules including surfactants (3-5), co-surfactant (6), and solvents like ethanoic acid (S) (Aswathanarayan and Vittal, 2019; Deshmukh et al., 2021). This nanoemulsion vehicle carries hydrophobic bioactives (7) dissolved in the oil phase.

The above-identified components of core-shell structure of nanoemulsion are engineered to have physicochemical interactions at the molecular level, and to assemble to form a stable architectural unit that can function as a drug carrier. Each of the above-described components have different physicochemical properties including the 3-D molecular shape, molecular size, electrical charges, that can influence (weakens or strengthen) the molecular assembly. In addition, the PH of the medium can influence the molecule orientation and assembly.

FIG. 2B depicts the morphology of single phospholipid supermolecule demonstrating a positively charged tail area which is lipophilic that can position itself in the oil phase (1) and a rounded negatively charged head area which is hydrophilic that can position itself in the water phase (2) (Li et al, 2015).

FIG. 3 depicts the types of microemulsion system classified according to the visual observation of the samples, i.e., clarity, transparency, and stability over time (Vavra et al., 2020; Goswami et al., 2016). Winsor classified microemulsion as four types: (i) Winsor Type I consist of two phases: the upper microemulsion phase and the upper excess oil in equilibrium; (ii) Winsor Type II consist of two phases: the upper microemulsion phase and the lower excess water in equilibrium; (iii) Winsor Type III where the middle microemulsion phase is in equilibrium with both the upper excess water and the lower excess oil; (iv) Winsor Type IV: this type is characterized by a single phase: the surfactant, water, and oil are all mixed homogeneously. Winsor IV-type behavior for up to 12 months at room temperature.

It needs to be clarified that, there is some confusion in the literature regarding a precise definition of nanoemulsions which are often confused with microemulsion, as nano have a short development history (Gupta et al., 2016; Seng and Loong, 2019). The similarities and differences in the physical and chemical properties between microemulsion and nanoemulsion are described in table 1.

FIG. 4 depicts a portion of the liposome lipid bilayer depicting its molecular assembly essentially consisting of phospholipid shell (1) encircling a water core (2) and is surrounded by the water phase (2). The surfactant molecules include phospholipid surfactant molecules (3), positively charged phospholipids (4), and negatively charged phospholipids (5), encapsulating lipophilic bioactive(s) within the lipid bilayer (7), also carrying hydrophilic bioactive(s) (8) in the water core and surrounding water phase and a water-soluble solvent (S).

II. Optimization process: Mix-and-match process.

A pseudoternary phase diagram is a tool used in formulation design and optimization of the heterogenous components of LBDDs, optimizing the three components of any typical emulsion, i.e., water, oil, and surfactant/co-surfactant mix (Smix), to obtain the concentration range of these components that forms a transparent stable emulsion.

FIG. 5A depicts ternary phase diagram depicting phase behavior of a mixture of oil and Smix during water titration. It is noticeable that, self-assembled, well-defined structures and patterns are spontaneously formed from oil, surfactant mix (Smix) and water at varying ratios and amounts during water titration that resulted into two types of emulsions, wherein (A) represents transparent nanoemulsion Winsor IV, and (B) represents milky emulsion Winsor type IV in different quantities.

FIG. 5B represents an example of pseudo-ternary phase diagram of anti-HIV drug Efavirenz-loaded flaxseed oil nanoemulsion where the three components of the system are plotted on the three corners of the triangle, with surfactant-mixture Ethanol: Tween® 80: Span® 20). Solutions of surfactants and oil, in all ratios for the surfactant mixtures are titrated in water and studied for visual appearance and stability. The diagram demonstrates that multiple types of LBDDs can result from different concentrations and ratios of its components, wherein (A) represents transparent nanoemulsion Winsor IV, (B) represents milky emulsion Winsor type IV, (C) represent milky Winsor I, II, & III, (D) represents translucent Winsor I, II & III, and (E) represents semisolid gel (Mazondi et al., 2020).

Construction of pseudoternary phase diagram is a multistep procedure that studies the impact of oil phase composition and surfactant concentration, and it include:
Determination of solubility of the drug in different oils, e.g., grapeseed, flaxseed, soybean, macadamia, and sunflower oil, and select the oil with the highest drug solubility to construct the diagram. Candidates are further examined for their emulsification properties;
The water titration method was used to mixtures along dilution paths. Solutions of oil and Smix in all ratios are titrated in water, and studied for Winsor type classification, visual appearance (i.e., transparency versus opacity and clarity versus turbidity), and stability over time (hours, days, or longer);
Statistical optimization D-optimal design is performed to study Smix optimization. The D-Expert software is used to elucidate the effect of the proportion of individual components of the surfactant mixture viz. Span 20, Tween 80 and ethanol content on the resultant emulsion, while maintain a fixed % of the oil.

Further, each spot on the diagram is called a centroid that represents a different composition of ternary components, and the mass fractions of the centroid can be read off from this triangle (Mazondi et al., 2020; Berkman and Gulec, 2021; Rosso et al., 2020; Chuesiang, P. et al., 2018; Patra et al., 2018; Shrestha et al., 2014).

The major disadvantage of ternary phase diagram is that it demands great number of experiments, is time consuming, and costly.

Challenges with current methods of production of LBDDs.

FIG. 6 is a flow chart that demonstrate the seven steps of the current manufacturing process of LBDDs as a carrier for certain bioactive phytochemical according to its specific physicochemical characteristics. Steps 1-4 were described above including: 1—Selection of the oil composition best dissolve the drug; 2—Selection of surfactant/co-surfactant mix and solvent that best matches the oil and the bioactive; 3—optimize the Smix to oil ratio using pseudoternary phase diagram water titration method; 4—the goal is to find the concentration and ratio of the components that produce a transparent clear and stable nanoemulsion Winsor type IV, and preferable the concentrations and ratios at the centroid point; 5—Now it is time to use the calculations made in the previous steps to manufacture the LBDDs by selecting the best suitable emulsification method.

The methods described in FIG. 6 include: high-energy, low-energy including low-energy phase inversion method that derives the emulsification energy from the components and solvent displacement, and the self-nanoemulsification method to manufacture self-nanoemulsifying drug delivery system (SNEDD). These methods are further described below:

Low-energy emulsification methods: Also known as physicochemical approach, uses the energy input from chemical potential of the components to form nanoemulsions. The low-energy methods involved in nanoemulsion production are phase inversion composition, phase inversion temperature, and solvent diffusion method. In this method the intrinsic physical properties of surfactant and the oil phase plays a major role in the product characters and how easily it can be scaled up, and the surfactant-to-emulsion ratio had to be optimized to produce fine droplets.

FIG. 7A depicts low-energy emulsification method involves breakdown of coarse oil-in-water macroemulsion into nanoemulsion. In this spontaneous emulsification process the oil phase (1) is mixed with water phase (2) that are meeting at the interfacial film (IF), in the presence of a surfactant/co-surfactant mix (Smix) that will promote low interfacial tension. Mixing the two phases with a magnetic stirrer (M) will result in the formation of a bicontinuous region (BI) and an oil-in-water coarse microemulsion (ME) will be formed; and finally, a thermodynamically stable fine nanoemulsion (NE) is produced.

The advantage of the low-energy method is that it involves minimal heat generation and thereby prevent the degradation of heat labile compounds. (Aswathanarayan and Vittal, 2019; Nor Azmi et al., 2019; Cheaburu-Yilmaz et al, 2019; Soni and Sharma 2021).

The major challenge of this method is related to the use of large amount of surfactant with side effects that impair the FDA approval, it requires full control of the physicochemical parameters, and the biggest problem is related to stability (the products are unstable, i.e., prone to coalescence and creaming). Thus, the method is not suitable for industrial-scale production of emulsions (Marzuki et al., 2019).

FIG. 7B depicts a low-energy phase inversion composition, wherein an oil-in-water emulsion is converted to water-in-oil emulsion by adding salts or even water.

FIG. 8 depicts high-energy emulsification method includes microfluidization, ultrasonication, and high-pressure homogenization. The microfluidization principle uses high energy to breaks macroemulsion drops (A) into nanodrops (E) being pumped through microchannels (B), while experiencing high shear forces and rupture under pressure of 500-2000 psi generated using positive displacement pump, and then passing through the interaction chamber (C), and the cooling jacket (D) to the outlet. (Tracey Nguyen, 2013; Gupta et al., 2016; Deshmukh et al., 2021). The process generates a lot of heat that might affect thermolabile compounds and requires expensive specialized microfluidizers. The microfluidization process was successful in producing mRNA vaccines for COVID-19. Another example is curcumin nanoemulsion in drug delivery and the food industry.

Recently, issues with nanoemulsions have been coped up by the emergence of self-nanoemulsifying drug delivery system (SNEDDS), which is an anhydrous form of nanoemulsion also known as emulsion preconcentrate (Plaza-Oliver et al, 2021). SNEDDS are isotropic mixtures of surfactant, oil, active pharmaceutical ingredient (API), and hydrophilic co-surfactant or co-solubilizer. Self-nanoemulsification occurs in within the gastrointestinal tract (Dilpreet Singh, 2021; Ur Rahman et al., 2016). Various preparative methods are available for SNEDDS, such as high-pressure homogenizer, microfluidization, sonication, phase inversion, and shear state methods. These methods show favorable benefits in drug delivery. SNEDDS possesses some disadvantages like precipitation of drug in G.I fluid or possible drug leaving in the capsule dosage form due to incompatibility issues (Dilpreet Singh, 2021).

Similarities and differences between different types of LBDDs.

LBDDs are classified into oil-in-water nano-micro-emulsion, water-in-oil nano-micro-emulsion, self-nanoemulsifying system, self-microemulsifying system, liposomes, micelle, and nanocapsule (Plaza-Oliver et al, 2021). There is some confusion in the literature regarding a precise definition of nanoemulsions which are often confused with microemulsion, as nano have a short development history (Gupta et al., 2016; Seng and Loong, 2019). The similarities and differences in the physical and chemical properties between microemulsion and nanoemulsion are described in table 1.

TABLE 1
Comparison of the physicochemical features of microemulsion versus nanoemulsion, and the processes used in their fabrication.

| Description ^{a-e} | microemulsion | nanoemulsion | Comments |
|---|---|---|---|
| Definition | Is a clear, stable isotropic | Is a special case of emulsion | |
| | liquid dispersion | | |
| Particle size | Between 10-100 nm/up to 0.15 | In the nanoscale/Between 10- | Particle size is a function of |
| | micrometer/100-200 nm | 100 nm/up to 0.25 micrometer)/ / | the ratio of the oil and surfactant |
| | | 100-200 nm | mix. Emulsion type differences cannot |
| | | | be distinguished merely from size ^{a} |
| Particle size distribution | Single narrow peak | Multiple peaks | |
| Thermodynamic stability | stable | Unstable | Phase separation and emulsion breakdown |
| | | | over time. Confers long shelf life |
| Kinetic stability | low | high | Gravitational stability is determined |
| | | | by particle size. |
| Optical properties/Light | Opaque or semi-transparent ^{b}/ | Transparent when the size is ≤30 | |
| scattering/appearance | Strong light scattering and | nm ^{b}/Strong light scattering, | |
| | transparent! | appearance may vary | |
| composition | An oil phase, water phase, | An oil phase, water phase, | |
| | surfactant and possibly co- | surfactant and possibly co- | |
| | surfactant, a greater surfactants- | surfactant | |
| | to-oil ratio is required | | |
| Fabrication methods | Principally, it can form | External high sheer forces must | Difficult to distinguish based on |
| | spontaneously | be applied | fabrication methods ^{b} |
| Method of preparation | Low-energy method | High-energy and low-energy methods | |
| Importance of order | Order of mixing does not matter | Only formed when surfactants | fabrication of nanoemulsion involves |
| of mixing | | are first mixed with oil ^{C; d} | specific mixing order in which, |
| | | | surfactant must be mixed first with |
| | | | oil phase, |
| Types | Direct, reversed, and bicontinuous | | |
| Applications | in some drugs and cleaning products | Pharmaceutical and food industry | |

References:
^{a}Koh and Wong, 2019;
^{b}Seng and Loong, 2019;
^{c}Ur Rehman et al., 2016;
^{d}Gupta et al., 2016; Lopes, Luciana, 2014;
^{d}Kale and Deore, 2016;
^{e}Sheth et al., 2020; McClements D., 2012.

Oil producing plants of medicinal value are plants from which oil can be extracted from flower buds, seeds, leaves, stems, or any other parts, and which are cultivated for the therapeutic, cosmetic, and recreational benefits of their oil. Examples of oil yielding plants are Nigella sativa, Cannabis and Hemp, Curcuma longa, and other plants that can be a uni-source for bioactive phytochemicals, oil phase components, and surfactants/saponins.

### Example 1: Nanoemulsification of Nigella Sativa to Produce Fermented Uni-Sourced Nigella-Loaded Nanocomposition

Background of Nigella sativa

Nigella sativa is an annual flowering plant in the family Ranunculaceae and genus Nigella that contains 20 species. In the current invention the family is represented by Nigella sativa, which is one of the eldest known medicinal herbs, found in Tutankhamun's tomb. Nigella sativa is also known as caraway, black cumin, habat albaraka, Kalonji, Ketzah, Schwarzkümmel, Hak Jung Chou. The FDA recognize Nigella sativa as “GRAS”, i.e., “Generally Recognized as Safe” (CFR—Code of Federal Regulations Title 21) (Mrozek-Wilczkiewicz et al., 2016; Padhye et al., 2008).

Nigella sativa oil composition and its bioactive phytochemicals.

Nigella sativa is an oil-producing medicinal herb, the oil of which includes the fixed and the volatile oil that encompasses the bioactive phytochemicals.

(1) Fixed seed oil of Nigella sativa is a source of essential fatty acids, glycolipids, phospholipids, and bioactive phytosterols, and showed high level of unsaturated fatty acids, cholesterol, sterols, and caryophyllene (Tiji et al., 2021; Piras et al., 2013; Gharby et al., 2015; Mohammed et al., 2016).

(2) Volatile oil of Nigella sativa is mainly composed by volatile components,

(3) Volatile components also known as Terpenes and Terpenoids or quinine, including thymoquinone (TQ) and thymoquinone derivatives (isomers) that includes dithymoquinone, thymohydroquinone, thymol, p-cymene, carvacrol, 4-terpineol, α-thujene, α-pinene, t-anethol, sesquiterpene longifolene. Other minor volatile components include carvacrol, β-pinene, limonene, methyl linoleate, and sabinene 4,5-epoxy-1-isopropyl-4-methyl-1-cyclohexene, and 4-terpineol (Klos and Chlubek, 2022; Abdul Hannan et al., 2021; Kabir et al, 2021; Tiji et al., 2021). Wherein, the versatility in the pharmacologic functions if Nigella sativa is due to the presence of thymoquinone.

(4) Saponins: Alpha-hederin is an important constituent of Nigella sativa is a water soluble pentacyclic triterpene and saponin, and also include_kalopanoxsaponin, nigella A-D) (Imran et al., 2022). Additional water soluble saponins (green solvents) from Nigella sativa including p-cymene, quercetin, steryl glucoside, and limonene.

(5) Alkaloids: Nigella sativa alkaloids are classified into isoquinoline alkaloids nigellicimine and nigellicimine-N-oxide, and pyrazole or indazole alkaloids nigellidine and nigellicine. In addition, alkaloid nigelamines A1-A5 proclaimed potent lipid metabolism-promoting activity (Mukhtar et al., 2020; Maideen N. M.).

(6) Water soluble bioactive phytochemicals: include alpha-hederin, and alkaloids that are water soluble under acidic conditions (Verpoorte R., 2005).

(7) Phospholipid emulsifiers: Nigella sativa phospholipids include (phosphatidylinositol, phosphatidylcholine, and phosphatidylglycerol, and glycolipids.

(8) Phytosterols: Oil extracted from black cumin contains several sterols, of which β-sitosterol is the main sterol and cholesterol that are effective natural agent in lowering blood cholesterol and preventing cardiovascular diseases.

(9) Flavonoids: including quercetin, kaempferol, rutin.

(10) Polyphenol family Being an antioxidant polyphenol, kaempferol helps prevent oxidative damage of cells and quercetin protects against various diseases such as osteoporosis, lung cancer, and cardiovascular problems. and platelet formation in the blood.

(11) Miscellaneous Components: Carbohydrates, proteins, minerals, and vitamins including Vitamin E are important emulsifier and natural antioxidants that scavenge free radicals and inhibit lipid peroxidation in biological membranes.

The two principal bioactive constituents of Nigella sativa are thymoquinone and alpha-hederin.

Thymoquinone (TQ) is the major bioactive phytochemical of Nigella sativa volatile oil that have a great biological potential. TQ is a monoterpene (C10H12O2), a hydrophobic compound that is extremely unstable in aqueous solutions due to the strong influence of pH and light. Consequently, the transition of TQ into clinical trials is being impeded by formulation issues and the route of administration. A viable solution to overcoming the roadblock to clinical translation is the formulation of TQ into a lipid-based drug carrier system, wherein the nano-Nigella and nano-TQ formulation can be used as an oral drug to treat diseases, a daily soft drink to enhance biologic activity, or a local cosmetic and topical medication (El-Far et al., 2018; Al-Gabri et al., 2021; Hannan et al., 2021).

Alpha-hederin is a triterpene saponin which have one or more hydrophilic moieties combined with a lipophilic triterpene or steroid derivative. Alpha-hederin is water soluble, stable under normal conditions, and appears as a white crystalline powder (Adamska et al, 2019). Alpha-Hederin has several biological properties such as antispasmodic, and inhibiting cell proliferation (Sigma-Aldrich), and has been identified as a potential anticancer agent. The intracellular location of alpha-hederin is within the seed coats and the inner seed tissues (Botnick et al., 2012).

Other plants alpha-hederin is found in other plants including Hedera helix, Chenopodium quinoa, Kalopanax pictus, that have anticancer properties.

Synthetic analogues of Nigella sativa bioactive phytochemicals.

The total chemical synthesis of thymoquinone analogues, nigellidine, nigellicine, nigellamine A2 is feasible (Khan and Afzal, 2016). In one embodiment, it is possible to add one or more of the synthetic analogues to the uni-sourced Nigella sativa nanocomposition to have a synergistic action of increasing its biological activity or bioavailability.

Current therapeutic indication for Nigella sativa

The pharmacological activity and indications for use of Nigella sativa and its nano-formulation can be divided into cosmetic (with special reference to its smooth muscle relaxing effect) and therapeutic, local, and systemic indications. Published research describe the use of Nigella ground seeds, the oil, aqueous and solvent extracts, nano-Nigella and nano-TQ formulations.

In the cosmetic field Nigella sativa have been used to formulate anti-aging products.

A. Smooth muscle relaxing effect: Nigella sativa have a relaxant effect on smooth muscles. There is evidence of the relaxant effects of this plant and some of its constituents on different types of smooth muscle including rabbit aorta, rabbit jejunum, trachea, and uterine smooth muscles. The relaxant effect of N. sativa could be of therapeutic importance such as bronchodilation in asthma, vasodilation in hypertension and therapeutic effect on digestive or urogenital disorders (Keyhanmanesh et al, 2014). Nigella sativa have a relaxant effect on tracheal smooth muscles of guinea pig (Bashir et al., 2020; Boskabady et al., 2011; www.mskcc.org; www.webmed.com; www.drugs.com; Koshak et al., 2017). Nigella sativa and its oil can be an adjuvant therapy for chronic obstructive pulmonary disease (Al-Azzawi et al., 2020). The compounds that are currently known to have smooth muscle-relaxing action are alpha-hederin and carvacrol that has a potent smooth muscle relaxant effect on guinea pig trachea. Both volatile and total Nigella sativa oils has a smooth muscle relaxant effect, as well as aqueous, crude, and macerated extracts. Thymoquinone has a quantitative relaxant effect on the tracheal smooth muscle (Bashir et al., 2020) and cardiac muscle relaxant effect (Ghayur et al., 2012)

B. The local use on the skin and scalp of nano-Nigella and nano-TQ are limited, mostly related to enhanced absorption (Al-Otaibi et al., 2018), treatment of psoriasis (Negi et al., 2019) and eczema, it enhances chemical penetration mostly the terpenes, and when combined with current drugs, i.e., tacrolimus (de Matos S P et al., 2019; Sudip and Koushik, 2021), and TQ-loaded chitosan nanoparticle as preservative with antibacterial activity in cosmetic products (Mondejar-Lopez et al., 2022). Black seed oil can also be applied topically. Small scale studies have demonstrated positive effects for eczema, psoriasis, atopic dermatitis, and acne (www.webmd.com/diet/black-seed-health-benefits#1).

C. The systemic use of Nigella sativa seed, oil, water extract, and other extracts in oral formulation have been studied as a possible alternative therapy or add-on therapy to many human diseases, Nano-TQ is the most effect component, wherein TQ effectively augment the anticancer role of doxorubicin in breast cancer cells. Nano-TQ protects against diabetes, inflammation, CNS, and hepatotoxicity mainly by enhancement of organs' antioxidant status. From the current studies, we can conclude that Nano-TQ is a promising nutraceutical for human health in the prevention and treatment of various disorders https://sciforum.net/manuscripts/10293/manuscript.pdf.

The use of Nigella sativa and its products (oil, nanocompositions) have been studied in the following diseases:

### Treatment of Metabolic Syndrome:

Metabolic syndrome is characterized by obesity, dyslipidemia and/or diabetes and its complications, and hypertension (U.S. patent application Ser. No. 17/291,545, titled “Composition comprising Nigella sativa oil and surface-active agents”; Dajani et al., 2018; El-Far et al., 2018). Online database about clinical trials “www.clinicaltrials.gov”, there are 16 studies on N. sativa beneficial effect on blood lipids, obesity, and diabetes management

### Anticancer Properties:

Nigella sativa has anti-proliferative, anti-angiogenesis, pro-apoptotic, anti-oxidant, cytotoxic, anti-mutagenic, and anti-metastatic effects, (Salehi et al., 2021). TQ and its nanoformulation prepared by double emulsion method was studied on colorectal and breast cancer cell line, and its protection against doxorubicin-induced cardiotoxicity (El-Far et al., 2021). Other studies of Nigella sativa essential oil nanoemulsion was conducted against cancer cell lines of human hepatocellular carcinoma; breast cancer; tongue (Abd Rabou A., 2021; Periasamy et al, 2016; Nirmala et al., 2020; Gomathinayagam et al. 2020). Nigella sativa volatile oil components include α-thujene, p-cymene and TQ are effective in liver cancer cell lines (Abd-Raboul and Edris, 2021). Nigella sativa is an effective anti-angiogenesis (Lei Peng et al., 2013).

Anti-COVID-19 property:

A multi-center placebo-controlled randomized clinical trial using Nigella sativa (80 mg. kg/day) and honey for up to 13 days, found significant improvement of symptoms and viral clearance in the patients compared to placebo (Ashraf S. et al., 2020, www.ClinicalTrials.gov, NCT04347382). Computer-assisted molecular docking identified nigellidine and alpha-hederin as novel inhibitors of SARS-CoV-2 (Salem and Noureddine; Hardianto et al., 2021; Abdelrahim et al., 2022). Nigellidine may inhibit SARS CoV-2 viral replication by strongly binding to spike protein at the hinge region acting on viral protein nsp3 binding-blocking effect (Banerjee et al., 2021). Nigellone (dithymoquinone) was reported as a strong inhibitor of COVID-19 in silico (Pandey et al., 2021), and reviewed by Fetian et al. (2020).

### Immunomodulatory Properties:

Enhancement of innate immune responses in the gut-associated lymphoid tissues and systemically by enhancing interferon secretion, T-lymphocytes, natural killer cells, and macrophage activities.

### Treatment of Respiratory Diseases:

Nigella sativa act as an asthma controller medication in asthma and allergic rhinitis in human clinical trial (USA patent #7,592,327, B2, 1999, by the current inventor), and chronic obstructive pulmonary disease. Alpha-hederin and TQ of Nigella sativa has preventive effect on sensitized rats, possibly by intervening in miRNA-126 expression, which consequently could interfere with IL-13 secretion pathway and IL-13 mRNA levels leading to a reduction in inflammation of lungs in ovalbumin-sensitized rats (Fallahi et al., 2016)

### Anti-Tuberculosis Activity:

The current inventor compared extracts of Nigella sativa to BCG [purified protein derivative of Bacillus Calmette Guerin (PPD)] using H3 thymidine incorporation and found that Nigella sativa extracts were identical to BCG (USA patent #7,592,327, B2, 1999); TQ inhibit Mycobacterium tuberculosis replication in macrophage cells and immune cell balance (Mahmud et al., 2017; Olivianto et al., 2021). Computer assisted molecular docking study of Nigella sativa against Mycobacterium tuberculosis found that alpha-hederin, diTQ, and nigellidine possess significant binding energy for RNAp of Mycobacterium tuberculosis (Mir et al., 2022). Nigella sativa has also been investigated for efficacy against anti-tuberculous drug-induced liver injury (Jaswal et al., 206).

Anti-inflammatory and analgesic effect:

In the treatment of Crohn's disease, facial palsy, inflammatory arthritis, degenerative osteoarthritis, and recurrent cystitis.

Antioxidant effect: is effective in the prevention of chronic and degenerative diseases associated with oxidative stress that is useful in the treatment of rheumatoid arthritis, diabetes mellitus, and hepatic injury.

Anti-viral effect: in the treatment of HIV, only few human case reports demonstrated that the administration of Nigella sativa produces seroconversion of viral load in HIV/AIDS patients (Onifadee et al, 2013; Onifadee et al., 2015; Maideen N., 2020). In the treatment of hepatitis C and B, Nigella sativa oil 450 mg softgel capsules was administered to 30 patients with hepatitis C (8 hourly for three months) resulted in significant decrease in HCV viral load (Baraket et al., 2013). Nigella sativa was investigated on HCV replicon cell line and was found inhibit viral replication (Oyero et al., 2016; Yang X Y et al., 2019).

Health-promoting effects in humans Effect on supplementation:

Clinical studies have mostly confirmed some effects of the seeds of N. sativa and their derivatives (seed extract and seed oil) obtained in the aforementioned in vitro and in vivo animal studies. The administration of N. sativa seeds (or seeds oil/extract) was alone or with other herbal substances (e.g., Curcuma longa extract), with different dosages and up to 60 days have demonstrated some benefits (Salehi et al., 2021).

### Other Indications:

Neuroprotective in central nervous system diseases including seizure, multiple sclerosis, Parkinson, Alzheimer's disease, and memory enhancement. A molecular docking of study of acetylcholine esterase enzyme can be inhibited by nigellidine, thus it can be a new option to treat Alzheimer's disease (Fouzia et al., 2019).

### Anti-Fungal Properties

Anti-bacterial activity: mainly against antibiotic-resistant bacteria.

Treatment of infertility and reproductive system diseases.

### BRIEF SUMMARY

According to embodiments of the present disclosure is a botanical uni-sourced lipid-based drug delivery nanocomposition loaded with bioactive phytochemicals of therapeutic benefits.

The botanical nanocomposition is composed of an oil phase and water phase with the components being arranged in a core and shell structure dispersed in a water phase containing surfactants that form and stabilize the oil phase nanoparticles in suspension.

The botanical nanocomposition is a drug carrying system for hydrophobic (lipophilic) unstable bioactive phytochemicals that are problematic during pharmaceutical formulation and storage, wherein the nanoformulation process of this invention extracts the volatile oil directly, drop-by-drop per second from their intracellular location, wherein the lipophilic bioactive is preloaded within the extracted volatile oil.

The raw material sources of the nanocomposition is one single oil producing plant with pharmaceutical and chemical interest in their bioactive phytochemical components, hence the novel tern uni-sourced nanocomposition, wherein selected plants include Nigella sativa, Cannabis sativa, and Curcuma longa.

The selected oil plants are the provider of the bioactive phytochemical (drug or Active Pharmaceutical Ingredient); volatile oil; fixed oil containing phospholipids, fatty acids; surfactants including saponins; carbohydrates; proteins; and electrolytes, that are all what is required in the composition of the uni-sourced lipid-based drug-loaded nanocomposition.

The solvent used in this process is any organic acid such as aqueous ethanoic acid at a concentration range from 0.01-50%, preferably 5%. The advantage of this solvent is that it has a dual function of extraction and emulsification, it can contain mother of vinegar for further fermentation, it is safe and palatable, it is not essential to remove it from the composition.

Incubation of the nanocomposition with the mother of vinegar results in the formation of a fermented uni-sourced lipid-based drug-loaded nanocomposition with an additional stability.

The process of the production of the nanoformulation is named the 4E process representing extraction, emulsification, ethyl alcohol fermentation, and esterification.

The process is further described as a “clock-wheel process” of optimization in the production of uni-sourced drug-loaded LBDDs from oil producing medicinal herbs, wherein:
(a) Select an oil producing medicinal herb for the production of the LBDDs that include at least one herb chosen from the group consisting of Nigella genus preferably Nigella sativa, Cannabis genus preferably marijuana and Cannabis sativa, and Curcuma genus preferably Curcuma longa;
(b) Add an aqueous solution of ethanoic acid to the grinded plant raw material and mix to start the extraction process;
(c) The medicinal herb provides an optimized amount and ratio of the oil and surfactant mix;
(d) The said optimization process provides a precise mixing order of the components including the volatile oil preloaded with lipophilic components forming the core; the fixed oil components forming the shell; surfactants dissolved in the water phase to stabilize the formed molecular assembly.
(e) The uni-sourced plant raw material provides a highly compatible components for improved molecular interactions and assembly;
(f) The said interconnected series of actions (extraction, dissolution, and emulsification) started and repeated over and over again in a self-controlled manner, with compatibility and precision are inherited in this process;
(g) The energy required for the emulsification process is ultralow and is derived from within the system.

### BRIEF DESCRIPTION OF THE SEVERAL VIEWS OF THE DRAWINGS

FIG. 1 is a flow chart depicting nanocomposition of current LBDDS product formulation including nanoemulsion, microemulsion and liposomes.

FIG. 2A depicting nanocomposition of multi-sourced nano-micro-emulsion with a core-shell structure demonstrating the shell with poor molecular assembly, fabricated according to methods known in the art.

FIG. 2B demonstrates phospholipid surfactant molecule with negatively charged hydrophilic head and a positively charged lipophilic tail which are known in the art.

FIG. 3 demonstrates Winsor classification of macroemulsion.

FIG. 4 depicting nanocomposition of current multi-sourced liposome demonstrating its phospholipid(s) bilayer with poorly assembled molecules.

FIGS. 5A and 5B demonstrates current optimization process of the multi-sourced components of LBDDs: Pseudoternary phase diagram.

FIG. 6 demonstrates current flow chart of the seven steps in current manufacturing process of LBDDs.

FIG. 7A demonstrates current low energy emulsification process converting macroemulsion into microemulsion and finally nanoemulsion.

FIG. 7B demonstrates current low energy chemical inversion process.

FIG. 8 demonstrate current sophisticated machine used in high energy emulsification process.

FIG. 9 is a flow chart of the method of forming a homogenous uni-sourced LBDDs formulation.

FIG. 10 demonstrates the composition of the novel uni-sourced nanoemulsion provided comprehensively from Nigella sativa.

FIG. 11 depicts the current invention process to produce a uni-sourced Nigella sativa nanoemulsion including the extraction and emulsification steps.

FIG. 12 demonstrates the chemistry of the biofermentation and esterification steps of the 4E nano-emulsification process.

FIG. 13 demonstrates the timeline of the 4E process and cumulative factors that stabilizes the uni-sourced LBDDs

FIG. 14 demonstrates the clock-wheel optimization process of Nigella sativa nanoemulsion formula.

FIG. 15 compares the sizes of Nigella sativa seed and the different grind sizes used in the preparation of LBDDs of this invention.

FIG. 16 depicting an example of the novel uni-sourced Nigella-loaded LBDDs nanocomposition demonstrating the molecular composition of the core and shell structure with a shell having tight molecular assembly.

FIG. 17 depicting an example of the novel fermented uni-sourced Nigella-loaded LBDDs nanocomposition demonstrating the additional surfactants added from the fermentation and esterification process to further stabilize the molecular assembly.

FIG. 18 demonstrates fermented uni-sourced Nigella-loaded liposome depicting the nanocomposition and tight molecular assembly of the phospholipid bilayer enclosing aqueous core incorporating components of fermentation and esterification.

FIG. 19 demonstrate coily hair follicle anatomy and physiology depicting the contracted “arrector pili” muscle as a pharmaceutical target for hair relaxation from the root using the first-in-class oral formula to relax the coily hair.

FIG. 20 depicting an example of the novel uni-sourced cannabinoid-loaded LBDDs nanocomposition demonstrating the molecular composition of the core and shell structure with a shell having tight molecular assembly.

FIG. 21 depicting an example of the novel fermented uni-sourced cannabinoid-loaded LBDDs nanocomposition demonstrating the additional surfactants added from the fermentation and esterification process to further stabilize the molecular assembly.

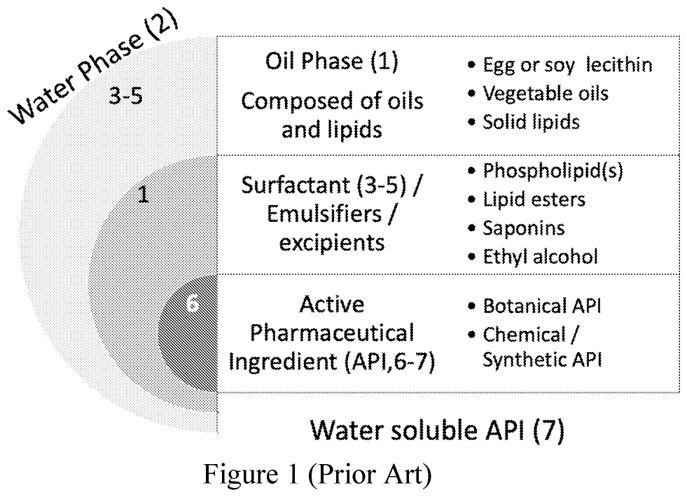

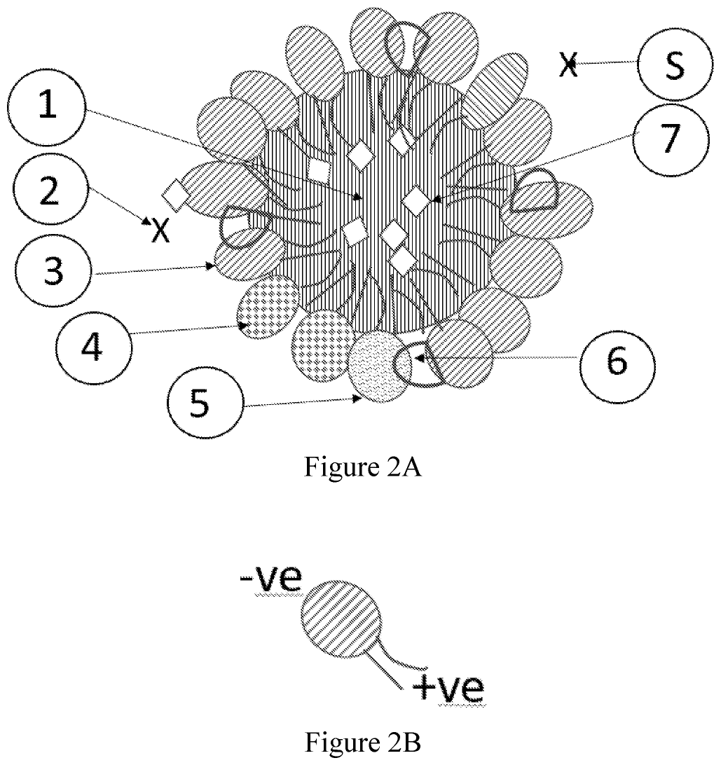

### DETAILED DESCRIPTION

According to embodiments of the disclosure is a novel botanical fermented uni-sourced drug-loaded lipid-based nanocomposition including a nanoemulsion product. The nanocomposotion is described characterized by using one single oil-producing medicinal herb as a comprehensive provider of all the drug-loaded nanoemulsion components and is described herein as “uni-sourced” nanoemulsion, wherein the selected medicinal herb can be an oilseed containing bioactive phytochemicals known for its pharmaceutical, cosmetic, or recreational benefits intended for oral and local administration, and wherein those bioactive phytochemicals have poor bioavailability and/or hydrophobic, unstable bioactives being problematic during formulation processes.

The chemical composition and the molecular assembly of the uni-sourced LBDDs products are described herein with the goal that a safe and stable nano-drug can be formulated, developed, and approved for use to treat humans in need for this drug and overcome the roadblock to commercialization.

Terminology used in describing the current invention: The design of the novel products of this invention is designated under the umbrella of lipid-based drug delivery systems (LBDDs) that includes nanoemulsion, microemulsion, liposomes, micelle, and drug lipid complexes and crystals. The term “nanoemulsion” is used interchangeably with LBDDs. In addition, he terms nanoparticle and nanodroplets are used interchangeably and could refer to any type of the LBDDs.

In the context of this description the word drug, bioactive phytochemicals, botanical active pharmaceutical ingredients (B-API), or API are used interchangeably.

Drug-loaded nanoemulsion and LBDDs refers to a drug delivery vehicle that can carry, protect, and deliver the hydrophobic and/or unstable bioactive phytochemicals to the target tissues inside the human in need for such treatment with the goal of enhancing the drug bioavailability.

In this invention, a formulation process of oil-in-water or water-in-oil botanical drug-loaded LBDDs is described, wherein the pharmaceutical formulation started by the addition of an aqueous phase contains an organic solvent preferably ethanoic acid, wherein the first step of the process is ethanoic acid extraction of the oil as nano-drops from its intracellular location, wherein the active drug is loaded in the oil prior to extraction; wherein water-soluble component of the oilseed including saponins and surfactants are solubilized in the aqueous phase. The tiny oil droplets extracted drop-by-drop will be spontaneously emulsified in the aqueous phase being mixed with the water-soluble surfactant/saponins alpha-hederin, phospholipids, P-cymene, and limonene. The hydrophobic sensitive drug molecule is loaded and protected within the core structure to get a beneficial medicinal product.

The process of the current invention includes the addition of an organic solvent to the grinded oil-plant or oil-seed; wherein any organic solvents currently used in the preparation of LBDDs can be used, including acetone, ethanol, hexane, isopropyl alcohol, and esters. In a preferred embodiment, ethanoic acid is selected to act as a solvent.

Process for formulation of uni-sourced LBDDs.

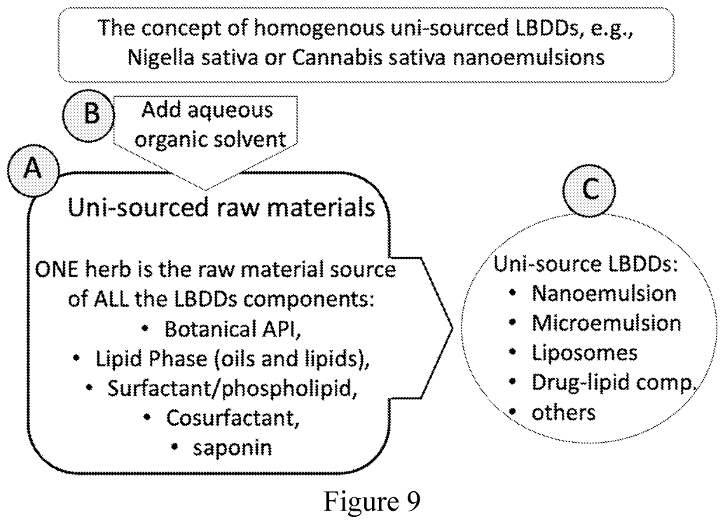

FIG. 9 is a flow chart that describe the concept and steps of the process to produce oil-in-water uni-sourced LBDDs that can be derived from either Nigella sativa medicinal herb or Cannabis sativa recreational/medicinal herb as an example of oilseeds that can serve as the one single source of the raw materials required to formulate LBDDs. The first step (A) is to grind required amount of the oilseed that is placed in an acid-resistant container; both Nigella and Cannabis sativa contains bioactive phytochemicals, oil phase components (oils and lipids), surfactant including phospholipids, co-surfactant, carbohydrate; protein; and terpene saponin (A). Second, is to add premeasured volume of aqueous ethanoic acid as an organic solvent (B) to the ground herb to initiate the process of extraction and emulsification with manual mixing, and then top up the container with the aqueous phase to produce different types of uni-sourced LBDDs including nanoemulsion, microemulsion, liposomes, micelle, or drug-lipid composition (C).

In one embodiment, the invention is related to Nigella sativa uni-sourced fermented nanoemulsion/microemulsion

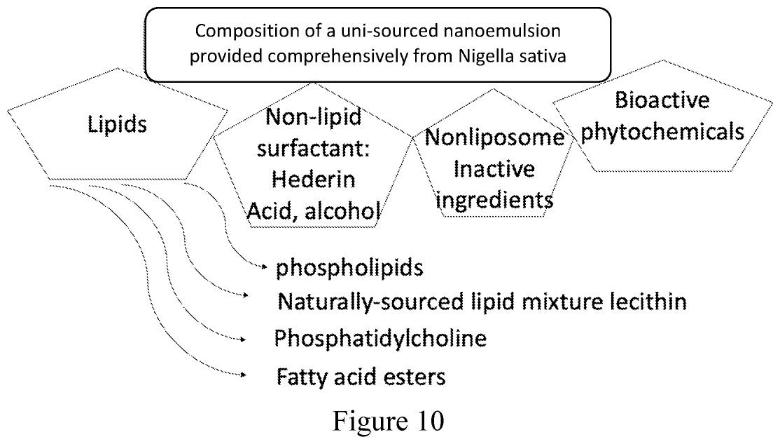

FIG. 10 demonstrates a flow chart of an embodiment depicting the composition of Nigella sativa uni-sourced nanoemulsion: As an example, the nanoproduct is composed of:

Bioactive phytochemicals both hydrophobic and hydrophilic (described in Table 3)

Lipids supplied including saturated (solid) and unsaturated fatty acids, phospholipids, naturally-sourced lipid mixture lecithin, phosphatidylcholine, and fatty acid esters

Non-lipid surfactants including alpha-hederin and ethyl alcohol

Nonliposome inactive ingredients.

The advantages of the invention extraction process are that it produces a clean formula free from insolubilized bioactive phytochemicals or surfactant, only what matters is extracted. The cake (meal) left behind after extraction of the oil phase and APIs from Nigella sativa is already separated with the unnecessary components. Another advantage is that This formulation can be compliant with FDA Guidance for the industry: Liposome Drug Product, April 2018, where the liposome drug product has a composition of 1), Drug substance, 2) Lipids i.e., naturally sourced complex lipids mixtures, 3) Nonlipid components of liposomes, and 4) Nonliposome inactive ingredients (e.g., buffer components with different concentrations and PH).

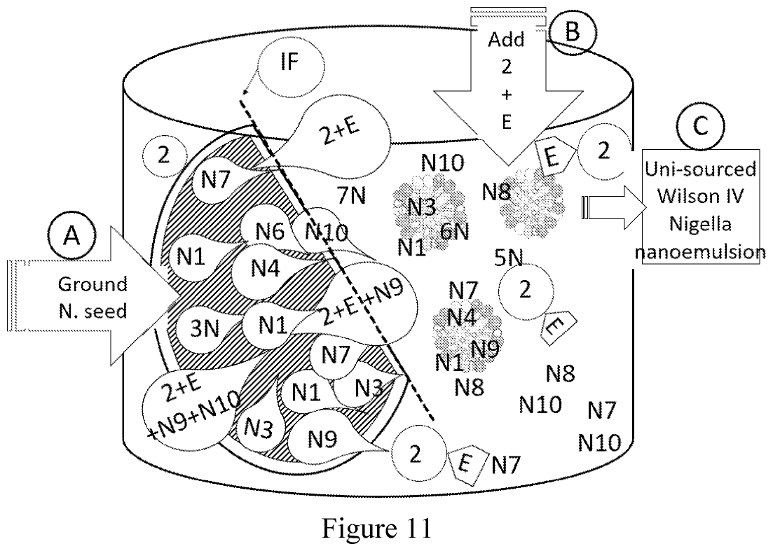

FIG. 11 depicts a stepwise description of Nigella sativa nano-emulsification process, wherein in step (A) a container with a known weight of Nigella sativa ground seeds (N) is used as the raw material and comprehensive provider of all the components of the nanoemulsion. In step (B) a measured amount of aqueous phase (2) is added to the ground seed and it contain the preferred solvent ethanoic acid (E) in a predetermined concentration, preferably 5%; Nigella sativa ground seed and the water phase interacts at the interfacial line (IF); aqueous ethanoic acid dissolves the cellulose of the cell membranes and extract the intracellular oil and lipids including the volatile oil (N1) that will form the core of the nanoparticle, Nigella phospholipids (N3); and the saturated and unsaturated fatty acids) (N4); wherein the oils and lipids are extracted drop-by-drop per second from its intravacuolar and intracellular locations; wherein dissolution of the water soluble Nigella saponin and green solvents, i.e., alpha-hederin, p-cymene and limonene (N5) and the Nigella proteins (N6) are happening simultaneously; wherein a liquid nanocomposition is being formed that encapsulate Nigella lipid soluble bioactive loaded into the oil phase (N7), e.g., TQ and Nigella water soluble bioactives (N8) e.g., alpha-hederin, nigellidine, nigellicine, nigellimine, nigellin, and melanthin. This drop-by-drop extraction in the presence of alpha-hederin and phospholipid surfactants facilitate the creation of ultra-low interfacial tension (IF) between the oil phase and water phase, thus allowing spontaneous emulsification and the formulation of oil-in-water, single phased, API-loaded uni-sourced Winsor type IV transparent stable Nigella sativa nanoemulsion (C) that can be used as pharmaceutical or cosmetic product.

The presence of electrolytes, carbohydrates, and proteins dissolved in the water phase increases the water phase density and facilitate the emulsification process and enhance the stability of the composition. Further, the saponification reaction is responsible for free fatty acid formation.

In anotherembodiment, a change in the aqueous solvent concentration % and the grinding size of the Nigella sativa seed will result in of oil-in-water, single phased, API-loaded uni-sourced translucent stable Nigella sativa microemulsion, liposomes, or micelles that can be used as pharmaceutical or cosmetic product

This novel process of extraction is converting the thick, dark Nigella oil that have very high interfacial tension with water into a water miscible oil that is emulsified to form a stable nano-micro-emulsion; a change that is explained in the nanopharmaceutical FDA guidelines described above.

Energy requirement: the process of the production of the uni-sourced Nigella sativa nanoemulsion is a low-energy process, wherein only minimal manual intermittent mixing is required in the first few hours, wherein the required energy for emulsification in ultralow and is derived from within the system.

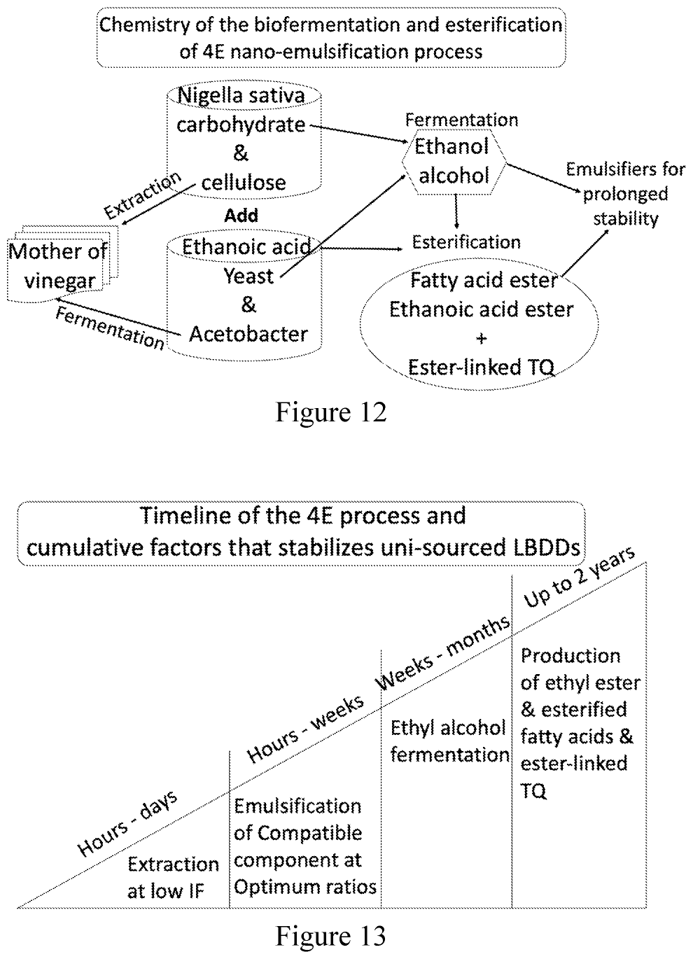

FIG. 12 demonstrates a flow chart of preferred embodiment of the current invention, wherein the composition resulted from extraction and emulsification of Nigella sativa using ethanoic acid as a solvent, as described in FIG. 11, is further incubated with “mother of vinegar” present in the commercially available unpasteurized aqueous 5% ethanoic acid solvent (E in FIG. 11). This incubation will result in a continuous biofermentation and esterification process of Nigella sativa carbohydrates and will generate ethyl alcohol (N9), ethyl ester (N10), and esterified fatty acids (N11) and ester-linked TQ. These cumulative factors (N9, N10, and N11) will make the nanoparticle shell molecular assembly tighter, and the nanoemulsion more stable for longer period of time.

The formation of mother of Nigella sativa.

In one embodiment, the incubation of the nanoemulsion in the presence of the mother of vinegar will result in the formation of the “mother of Nigella sativa”, which is a thick gelatinous opaque yellowish biofilm disc formed on the surface of the liquid composition composed of cellulose and acetic acid bacteria that feeds on fermentation products. It grows in size and form multiple layers. It can be formed under both anaerobic and during aerobic incubation. The mother of vinegar is formed when we use the ethanoic acid solvent that is unpasteurized and contains the mother of vinegar, and the cellulose is produced by the action of ethanoic acid dissolving the cellulose contents of the cell wall of Nigella sativa while extracting the seed oil.

The fermented uni-sourced LBDDs: Chemical equations of fermentation:

Nigella sativa seed contains carbohydrate of about 20-40% of its weight. During the incubation of the composition in the presence of mother of vinegar (yeast and acetic acid bacteria) ethyl alcohol will be produced, as described in the equation:

Yeast+glucose=ethanol(ethyl alcohol)(N9) or butanol(butyl alcohol)+carbon dioxide

The formation of ethyl alcohol is advantageous, it can act as a surfactant to stabilize the shell molecular assembly.

The esterified uni-sourced LBDDs: Chemical equations of esterification:

In the esterification process, both ethanoic acid (E) and Nigella sativa free fatty acids and can be esterified:

Carboxylic(ethanoic)acid+alcohol=an ester+water+gas bubbles

Free fatty acids+alcohol in acid-catalyzed esterification=fatty acid esters+water+H2gas bubbles

In addition, an ester-linked TQ is being formed.

The advantage of the fermentation and esterification step is that it produces additional “surfactants” or stabilizers, i.e., ethyl ester (N10) and esterified fatty acid (N11), that are important in long-term stabilization.

During the incubation period for both fermentation and esterification chemical reactions a white and black salt-like precipitate is formed at the mouth of the container, that builds up with time. This can be explained as the compound is formed and being oversaturated in the liquid composition.

FIG. 13 is a flow chart that describe the timeline of the process of extraction; emulsification; ethyl alcohol fermentation; and esterification (4E process), wherein mixing predetermined weight of grounded herbal seed with the aqueous ethanoic acid 5%, with intermittent manual agitation initiates an immediate process of extraction of Nigella sativa oil, lipophilic APIs and hydrophilic components; creating favorable quantities and ratios of the components and solvents that allows immediate oil droplet emulsification in a continuous process that can last for few days and up to few weeks. Wherein, incubating the composition for few weeks with the mother of vinegar will start the process of fermentation of Nigella sativa carbohydrates forming ethyl alcohol for a period that can range from weeks to few months; and further in time esterification of ethyl alcohol, free fatty acids, and the formation of ester-linked TQ will continue, and the process is described as a continuous bio-process.

The advantages of this 4E processes are that it creates additional co-surfactants (N9-N10) to stabilize the nanoemulsion and extends its shelf live for 1-2 years.

Diffusion-controlled spontaneous emulsification technique: The process describes above is a diffusion-controlled spontaneous emulsification technique which allows the formation of nanoemulsion and other LBDDs with minimal external energy input, wherein the required energy for emulsification in ultralow and is derived from within the system. The spontaneous emulsification process is facilitated by the presence of spontaneously adjusted surfactant concentration extracted from the seeds and dissolved in the water phase. The spontaneousity of the emulsification process is influenced by the surfactant structure, concentration and initial location, oil phase composition, addition of co-surfactant and non-aqueous solvent, as well as salinity and temperature (Zhe Li et al., 2020). This phenomenon is triggered by gradients of chemical potential between the phases (Solans et al., 2016). first step of spontaneous emulsification is described as interfacial turbulence, diffusion, and stranding, negative interfacial tension (Davis et al., 2021; Akram et al., 2021).

Drug loading in the uni-sourced emulsification process: In this invention, the hydrophobic (lipid soluble) Nigella sativa bioactive phytochemicals, e.g., TQ, is dissolved and preloaded in the volatile oil of the seeds prior to emulsification, hence it is encapsulated within the oil core of the nanoemulsion with high efficacy and minimum wasted API. The hydrophilic bioactives dissolve in the water phase and are part of the nanoemulsion formula, and can serve different functions within the nanoemulsion, e.g., alpha-hederin saponin as a surfactant and a drug.

Making an emulsion.

The process of emulsification of two immiscible liquids involves optimizing the selected components; making small oil droplets that are dispersed in the aqueous phase; and stabilizing them by adequately coating each droplet with the appropriate emulsifier.

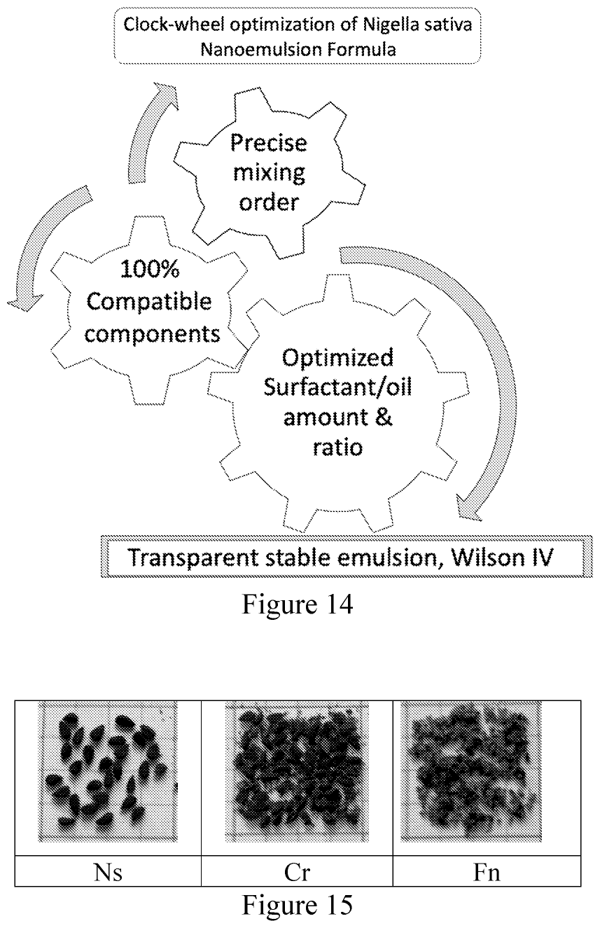

FIG. 14 is a flow chart that depicts a novel process of clock-wheel optimization in the production of uni-sourced lipid-based drug-loaded nanoemulsion from oil producing medicinal herbs, wherein an interconnected series of actions (extraction, dissolution, and emulsification) started and repeated over and over again in a self-controlled manner, with compatibility and precision are inherited in this process. Three variables were considered as important process parameters: optimized oil/surfactant amount and ratios, precise mixing order, and 100% compatibility of the components.

The clock-wheel optimization process is started by mixing the aqueous solvent/emulsifier and the grinded plant material; wherein the amount of the oil extracted is determined by the rate at which aqueous ethanoic acid penetrates into the intracellular and vacuolar spaces of the herbal seeds; and the said penetration is simultaneously associated with dissolution of the hydrophilic components of the herb entering the aqueous phase in an amount responsive to demand per unit of time (minutes or hours); hence, their ratios at certain point of time is precise and self-controlled.

The mixing order is precise and self-controlled, wherein the volatile oil is preloaded with the bioactive phytochemical; wherein the volatile oil is extracted as droplets from within the fixed oil which contains the surfactants; wherein the surfactants stabilize the said extracted volatile oil droplets; wherein the said oil droplets are dispersed in the aqueous phase that contains hydrophilic surfactants. mixing order is a robust factor that favours the production of a nanoemulsion with optimum physicochemical characteristics, e.g., transparent, single phased, stable nanoemulsion (Winsor type IV). It also favours the production of microemulsion, liposomes. Different types of LBDDs are formed under conditions when the acid concentration is changed.

Component compatibility is almost 100%, as the components are derived from one single plant source; these components agree with each other to a large extent reflecting the closeness between them; wherein all the components of the LBDDs are designed to interact and assemble at the molecular level forming a stable and functioning drug carrier.

Materials and methods for preparation of uni-sourced Nigella sativa LBDDs.

Examples of the materials used for the preparation of Nigella sativa uni-sourced LBDDs:

Nigella sativa seeds that can act as a comprehensive source of all the components of the said nanocomposition and will be detailed within the description of the figures.

A solvent for the extraction of the plant oil from its intracellular location within the plant cells. Selected solvents are the water-soluble organic acids and in some embodiments carboxylic acids, in one example embodiment ethanoic acid aqueous solution is used as a solvent, possibly in range from 0.01 to 50%, such as in 5% aqueous solution. The ethanoic acid can be either raw unpasteurized containing live mother of vinegar, pasteurized, or it can be distilled vinegar.

Water used can be either natural spring water containing electrolytes or distilled water.

Nigella sativa seed grinding sizes.

The seeds can be used whole, or in a preferred embodiment the seeds are grinded.

The grinding sizes can either very coarse, coarse, fine, or very fine. Other parts of the plant can also be grinded and used including the roots, shoots, or whole plant.

FIG. 15 demonstrate the size of Nigella sativa seed placed on one-inch graph paper to compare the sizes of ground Nigella sativa seeds in this experiment, wherein the whole seed (Ns) is compared to a coarse ground seed (Cr), versus finely ground seed (Fn). The length of Nigella sativa said is 1-5 mm.

Examples of the method used for the preparation of Nigella sativa LBDDs:

Addition and mixing steps: 200-400 grams ground seeds are placed into airtight containers resistant to the acid, preferably glass containers with a known volume, e.g., about four liters; add the first volume of one liter 5% aqueous ethanoic acid that is called the extraction liquid; mix by manual shaking or using a wooden spatula at short intervals (5-20 minutes) and keep for few hours (1-8 hours) which is the extraction time; this is followed by the addition of three liters of emulsification liquid that can be either water (resulting in 1.25% acidity of the final composition), 5% aqueous ethanoic acid or a mixture of 1 liter 5% aqueous ethanoic acid plus 2 liters of water (resulting in 2.5% acidity of the final composition); mix as above for few times (4-10 times); close the container air tightly and incubate at room temperature, within a range from 37-130° F. the composition is ready for use from day 1 and can be stored up to 1-2 years unchanged.

The selection of this room temperature range is critical to avoid heat induced denaturing of the natural compounds.

In a preferred embodiment, incubation is done in day light or lamp light, but it can be incubated in a dark room.

Incubation can be done under aerobic condition, and in the Prescence of “mother of vinegar” a Nigella sativa mother of vinegar is formed, that can be used as a product for use to treat humans in need for indications detailed in this invention.

The extraction and emulsification process can be conducted in a wooden barrel used for alcohol or vinegar fermentation or any suitable containers for food industry.

Which type of Nigella sativa uni-sourced LBDDs product is being formulated?

Table 2 demonstrate the three factors that can determine which type of LBDDs, and stability of the product being formed during the diffusion-controlled spontaneous emulsification process:
Concentration of ethanoic acid % in the emulsification liquid
Grinding size of the herbal seeds
Weight of Nigella sativa herb in the total volume. Seeds in the same family bought from different suppliers showed no differences that can be visually determined

TABLE 2
demonstrate examples of different LBDDs products prepared by changing the
grinding size and the acid concentration of the emulsification water phase.

| | Extraction | | | |
|---|---|---|---|---|
| N. sativa | liquid volume, | Emulsifying | Total vol. | LBDDs |
| grinding size | acidity % | liquid type | acidity % | Product type |
| coarse | 1 liter, 5% EA | 3 liters water | 1.25% | Transparent nanoemulsion |
| | (ethanoic acid) | | | yellow, one phase, stable |
| Coarse | 1 liter, 5% EA | | 1.66 | Transparent nanoemulsion |
| | | | | yellow, one phase, stable |
| Coarse | 1 liter, 5% EA | 3 liters ethanoic | 5% | Translucent microemulsion |
| | | acid 5% | | milky, one phase, stable, |
| Coarse | 1 liter, 5% EA | 3 liters ethanoic | 5% + | Within weeks or months white |
| | | acid 5% | anaerobic | (and black) precipitate will |
| | | | incubation | form at the container top |
| fine | 1 liter, 5% EA | 3 liters ethanoic | 5% | Translucent microemulsion |
| | | acid 5% | | milky, one phase, stable, |
| fine | 1 liter, 5% EA | 3 liters ethanoic | 5% | Translucent microemulsion |
| | | acid 5% | | milky, two phase, unstable |
| | | | | (separation into two phases |
| | | | | with time) |
| Very fine | 1 liter, 5% EA | 3 liters ethanoic | 5% | Milky opaque granular |
| | | acid 5% | | liposome, one phase, stable |
| Whole seed | 1 liter, 5% EA | 3 liters ethanoic | 5% | Black liquid one phase stable |
| | | acid 5% | | |
| Commercial | 5% EA equal | / | / | Immiscible, shiny crystals |
| oil | volume | | | within the EA, stick to the |
| | | | | jar wall and in the bottom |

Extraction in an acidic medium.

Table 3 demonstrate the PH measurement of the samples of nanoemulsions and microemulsions compared to the raw material used in their preparation. The advantage of acid PH is that TQ is stable at acidic medium, and alkaloids are water-soluble in acidic medium. The solution PH readings was taken using Dr. Meter brans model PH-100, with a measuring range of 0.01-14.00 with 500 error.

TABLE 3
demonstrate PH readings of Nigella sativa uni-sourced nanoemulsion
extracted/emulsified by ethanoic acid, compared to Nigella sativa
water extract, spring water and commercial ethanoic acid solvent

| Commercial ethanoic | | Ns water extract |
|---|---|---|
| acid (Vinegar) | Spring water | 200 gm/2 liters |
| 3.21-2.95 | 7.5 | 5.92-6.1 |

| Nanoemulsion clear | Microemulsion translucent |
|---|---|
| Sample 1 reading: 4.35-3.84 | Sample 3 reading: 3.57-3.47 |
| Sample 2 reading: 3.84/3.92 | Sample 4 readings are: 3.5-3.49 |
| | 3.84/3.91 Semi-translucent |
| | (small July sample Crescent 1/4) |

Foot note of table 3: All readings below 7 is acidic. The smaller the PH reading # the higher the acidity. Water is neutral.

Extraction/emulsification temperature.

The process can be done at room temperature of ranging from 59-86° F. (15-30° C.). although it can be carried out at any temperature used in laboratory procedures and pharmaceutical storage.

What are the components of the uni-sourced Nigella sativa LBDDs?

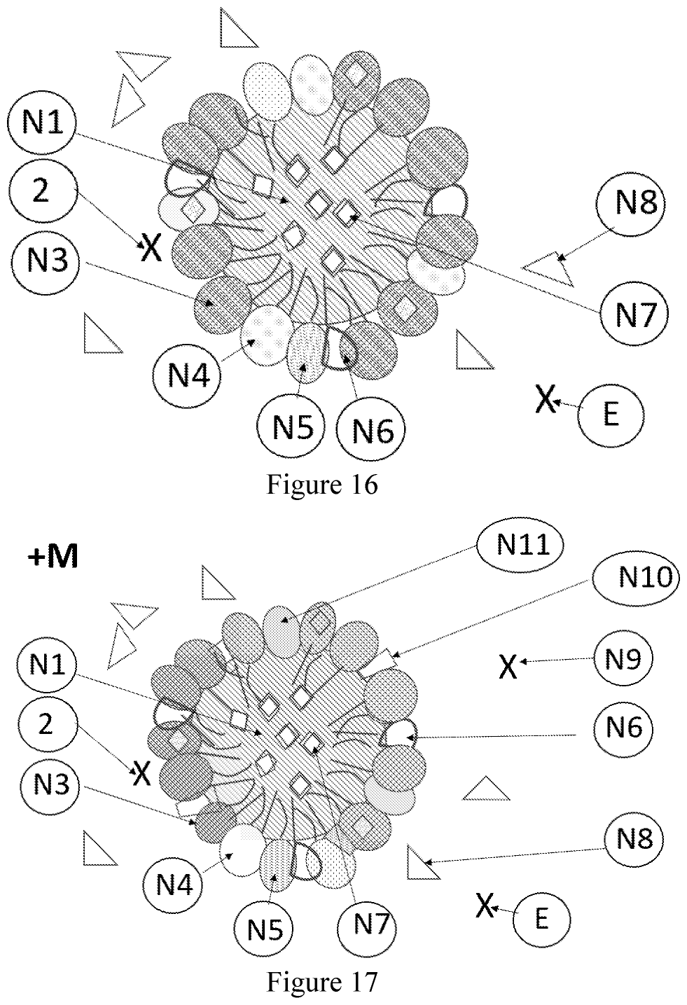

FIG. 16 demonstrates an example of the components of uni-sourced Nigella-loaded LBDDs nanoemulsion and their molecular assembly, wherein the uni-sourced nanoemulsion/microemulsion is consisting of nanoparticle (nanodroplet) assembled as a core and shell; wherein the nanoemulsion is composed of an oil phase (N1) and a water phase (2) containing aqueous ethanoic acid solvent (E). The letter (N) indicate that the components are derived from the herb Nigella sativa Wherein the core represents the oil phase (N1) of the particle which is composed of Nigella sativa volatile oil; wherein the shell is composed from other components derived from Nigella sativa oil including phospholipid (N3), saturated and unsaturated fatty acids and cholesterol (N4), and Nigella proteins (N5); wherein Nigella sativa saponins (N6), mainly alpha-hederin, are also part of the shell components. The lipophilic Nigella bioactive components are preloaded in the volatile oil core mostly thymoquinone (N7) and the hydrophilic bioactive compounds mostly alpha-hederin (N8) are dissolved in the surrounding water phase.

In the current uni-sourced extraction/emulsification process all the following components are derived from Nigella sativa including: the major lipophilic bioactive phytochemicals from Nigella sativa are TQ and TQ analogue; wherein TQ is being dissolved in the volatile oil prior to extraction and will be loaded in the particle core within the oil phase. The ester-linked TQ formed during the esterification process will be loaded to the nanoparticles at a later stage of the 4E process. Other lipophilic components of Nigella sativa are loaded in a similar manner.

The hydrophilic bioactive phytochemicals of Nigella sativa including alpha-hederin, quercetin, steryl glucoside, pyrazole alkaloids (i.e., nigellidine and nigellicine), isoquinoline alkaloids (nigellicimine and nigellicimine-N-oxide), and polyphenolic compounds (flavonoids), short chain fatty acids and sugar contents (Babar et al., 2019). Alpha-hederin is a triterpene saponin which have one or more hydrophilic moieties combined with a lipophilic triterpene or steroid derivative with anticancer properties. In this process, the saponin α-hederin is extracted from its accumulation sites, both in the seed coats and the inner seed tissues at different ratios (Botnick et al., 2012). Saponification reaction is responsible for free fatty acid formation.

Nigella sativa volatile oil have antioxidant activity, hence there is no need to add an antioxidant to the nanocomposition, the antioxidant activity of Nigella sativa L is more attributed to flavonoids and polyphenols than fatty acids (Tiji et al., 2021).

What are the components of the fermented uni-sourced Nigella sativa LBDDs?

FIG. 17 demonstrates a preferred example wherein the unisourced nanocomposition is fermented, wherein the uni-sourced nanoemulsion is consisting of nanoparticle (nanodroplet) assembled as a core and shell and is composed of the components N1-N8; wherein the product is further fermented by the addition of mother of vinegars (M); wherein the fermentation process of uni-sourced Nigella LBDDS will convert the carbohydrate components in acidic medium and result in the production of Ethyl alcohol (N9); wherein further esterification process of uni-sourced Nigella LBDDS will result in the production of Ethyl esters (N10) and esterified fatty acids (N11) incorporated in the shell resulting in tight molecular assembly for improved carrier function.

In other embodiment the nanoparticle core consist of multiple oil droplets, rather than a single droplet.

FIG. 18 depicts a segment of the shell of uni-sourced Nigella sativa liposome; where the differences in the solvent concentration and the Nigella seed grinding size will result in a milky opaque granular composition, suggestive of a liposomal type LBDDs. The liposome is a vesicle with a shell composed of phospholipid bilayer incorporating the oil phase (N1); the bilayer is enclosing a water droplet core and dispersed in an aqueous phase (2). The shell is composed of phospholipid bilayer (N3); wherein the molecules of each layer are aligned in one direction with the oil phase (2) being sandwiched in between the bilayer; wherein the phospholipids incorporate positively charged phospholipids (N4) and negatively charged phospholipids (N5) and cholesterol. The other components are similar to FIGS. 16 and 17.

In other embodiment currently available commercial Nigella sativa oil will serve as the uni-source of the raw material, where in the commercial Nigella sativa oil contains lipophilic bioactive phytochemicals, oil phase components (oils and lipids), surfactant including phospholipids only; In other embodiment, when the oil is mixed with the ethanoic acid in the high range of 2.5% to 5% or above the oil continue to be immiscible with the water phase that contains ethanoic acid even with agitation due to the lack of Nigella sativa water-soluble saponins (e.g., alpha-hederin). This process of mixing the commercially available Nigella sativa oil with aqueous ethanoic acid resulted in the formation of crystals (nanocrystals) that floats in the aqueous phase, deposited at the bottom of the container, or stick to the container walls.

In one embodiment, after extracting only what is needed, he Nigella sativa cake leftover contains many of the constituents that have many bioactive that can be recycled in the same process, used as animal feed or plant food.

In other embodiment the process of this invention is used to produce LBDDs from other oil producing medicinal herbs containing bioactive phytochemicals can be extracted/emulsified and possibly further fermented and esterified using ethanoic acid without or with the mother to produce uni-sourced LBDDs. Examples of oil medicinal herbs are Curcuma longa, ginger.

Advantages of the 4E Nigella sativa nanoemulsification process.

There are numerous advantages of the extraction, emulsification process of this invention, including:

No heat is used that will affect thermolabile and unstable volatile constituents.

No need for drying of the plant, thus preserve THS that can be lost during drying of the plant.

A clean formula is produced, free from unsolubilized bioactive phytochemicals or surfactant, only what matters is extracted! and the cake (meal) left behind can be separated by simple centrifugation.

A safe formula the overcomes the need for surfactants with side effects.

The solvent used is ethanoic acid that have multiple advantages:

(1) unique dual function of solvent and emulsifier.

(2) Ethanoic acid has the advantage of being GRAS “Generally Recognized as Safe”.

(3) Have acceptable taste and odor; hence, it can be retained in the composition. Alternatively, the taste and smell of ethanoic acid can be minimized by additives known in the art, or by hierarchical membrane dialysis(Quin et al., 2016).

(4) Ethanoic acid extraction process has the advantage of reducing the color impact of chlorophyl, improving, and softening the taste, bitterness, and aroma of Cannabis to make it more palatable for oral formulation.

(5) The ability of the process to continue in the presence of the “mother of vinegar”, that will produce fermentation and esterification products, from within the system, that will enhance the nanoemulsion stability and function.

This formulation can be compliant with FDA Guidance for the industry that describe the components of a liposome (LBDDs) as: 1), Drug substance, 2) Lipids i.e., naturally sourced complex lipids mixtures, 3) Nonlipid components of liposomes, and 4) Nonliposome inactive ingredients (e.g., buffer components with different concentrations and PH (Liposome Drug Product, April 2018).

The process is suitable for large scale industrial manufacturing because of easy repeatability, long-term stability of the products, low cost, and low energy requirements.

Improved LBDDs composition that mimic the composition of a living cell wall, as described below.

Improved LBDDs composition that mimic the composition of a living cell wall.

The improvement in the function of current uni-sourced nano-Nigella and nano-TQ is based on the striking resemblance between the components of a living cell wall and products of the current invention.

As demonstrated in table 4, the components of human cell membrane are compared to the components of Nigella sativa vs current liposome contents to show the striking degree of resemblance vs differences. NS liposomes have a component the better mimic the constituents of a human cell membrane with double layer of phospholipids. The goal of this design is to improve its functional characteristics, and to use 100% natural lipids and avoiding synthetic lipids.

TABLE 4
demonstrate the close similarity of the lipid components of a living cell wall
to Nigella sativa composition, compared to current liposome composition.

| Lipid Bilayer | | | |
|---|---|---|---|
| Molecular composition | Cell membrane | Nigella sativa | Current Liposomes |
| Phospholipids (PLs) | Yes, 50% of cell | Yes, 37% of total | Yes, Mostly synthetic |
| | membrane | lipid. Natural | or semisynthetic (FDA |
| | | | Guidelines, 2018) |
| Phosphatidylcholine (PC) | Major PL | Major class | Yes Sourced from egg or |
| (With neutral charge) | | | soy (permeable, less stable) |
| Phosphatidylethanolamine | Major PL | Major class | No |
| (with neutral charge) | | | |
| Phosphatidylserine | Major PL | Major class | No |
| (target macrophage, with | | | |
| negative charge) | | | |
| Phosphatidylinositol | Small amount | Major class | No |
| Phosphatidylglycerol | | Yes | Yes |
| (Target macrophage) | | | |
| Dipalmitoylphosphatidylcholine, | | | Yes |
| Distearoylphosphatidylcholine | | | |
| (saturated, rigid, impermeable), | | | |
| Stearylamine, | | | |
| dicetylphosphat | | | |
| Sphingomyelin | Major PL | No | Yes (in one type only) |
| (with neutral charge) | | | |
| Hydrogenated Lecithin | Yes | No | Yes, derived from egg or |
| (natural emulsifier) | | | soybean (Azad et al, 2020) |
| Cholesterol | Yes | Yes (Matthaus & | Yes |
| (and other sterols) | | Ozcan, 2011 | |
| Glycolipids | Yes | Yes (Ramadan & | No, Only in liposomes |
| | | Morsel, 2003) | with modified surface |
| Fatty acids incorporated | Yes | Yes, Palmitic and | No |
| in the phospholipid cell | | Stearic (saturated) | |
| membrane saturated and | | | |
| unsaturated | | | |
| protein | Yes (50%) | Yes | No |
| Main reference | Albert et | Ramadan and Morsel, | Leitgeb et al, 2020 |
| | al, 2002 | 2002 | |

When the Uni-sourced LBDDs product can be used?

The composition is ready for use within 1 day after its formulation, and anytime thereafter may be weeks. The fermented uni-sourced Nigella-nanocomposition or TQ-nanocomposition can be used within few weeks, months, or years.

The dosage of oral and local formulation.

The dosage of oral formulation is based on Nigella sativa single dose recommended for use in humans, and is calculated as:

The weight of extracted Nigella sativa seeds in the container/volume of the container X grams of Nigella sativa dose=volume of the emulsion single dose in CC.

A dosage of 1-3 grams/day was found to be effective, but any dosage between 0.5-20 gram/day can be used. The dosage is not a limiting factor of the invention.

In other embodiment a single oral dose is calculated according to the bioactive phytochemical compound concentration in the formulation, that can be based on the dosage of the oil can range from 20-150 mg/day

The dosage of TQ in children clinical trial was 1.0 mg/kg every 8 hours Dajani et al., 2018).

Alpha-hederin administration in a dose of (0.02 mg/kg) in ovalbumin sensitized rats as asthma model (Nazrul Islam et al., 2020).

The duration of treatment depends on the disease condition being treated ranging from short courses of one day to one week, or a long course up to few weeks or months. The duration of treatment is not a limiting factor of the invention.

Local therapy as intranasal spray, inhalation, skin application and scalp application are calculated according to the concentration of the bioactives in the liquid product. The application can be repeated multiple times per day for a duration of days, weeks, or longer according to the medical condition and the patient needs.

Overcoming the roadblock to approval and marketing.

The material used in this extraction/emulsification process are safe for human consumption. The process of production of the current invention has the advantage of extracting “only what matters” out of the complex compositions of the plants used as a source for the LBDDs components, i.e., it has simple LBDDs product composition! That will facilitate the approval process of the novel products as a nanodrug. An example of a liposomal formulation as described by the FDA was described in the background section. Upscaling of the process for industrial production is feasible, no need for solvent removal, no heating that might damage the thermolabile components.

How many products of Nigella sativa uni-sourced LBDDs nanocomposition can be produced?

In anotherembodiment, the nanocomposition of the current invention can be formulated into a liquid oral dosage form contains the bioactive phytochemical loaded into a LBDDs. The oral dosage form might include liquid emulsion, suspension, solution, elixir, and syrup.

In other embodiment, the nanocomposition can be formulated into gummies, liquid-gel capsules, beverages, dietary supplement, or other formulations known in the art. The liquid oral dosage form can be further classified according to their physicochemical properties into nanoemulsion (transparent and clear), microemulsion (translucent and clear), and particulate (milky and opaque) including liposomes or micelle.

In other embodiment, the oral formulation of Nigella sativa LBDDs can be combined with other available synergistic drug to improve its clinical outcomes and therapeutic benefits.

In other preferred embodiment, a topical preparation is formulated as liquid, or cream, lotion, gel, or ointment known in the art.

The products can be classified according to the chemical composition into either Nigella sativa uni-sourced LBDDs or fermented uni-sourced LBDDs; further, products can be divided into either Nigella-LBDDs or TQ-LBDDs, depending on the characterization of the major bioactive phytochemical loaded and encapsulated within the oil phase, and possibly what is dissolved in the water phase,

Additional products include the white and black precipitate, the mother of Nigella sativa, and the shiny crystals.

An examples of a product descriptive nomenclature are uni-sourced TQ-nanoemulsion, fermented uni-sourced TQ-nanoemulsion, fermented uni-sourced TQ-microemulsion, etc.

All of the above products will be named as “fermented Uni-sourced Nigella-loaded LBDDs” in further discussion and in the claims.

Formulation of Nigella sativa products of this invention might need the addition of coloring, flavoring, PH adjusting, fillers, stabilizers, excipients, or any other additives known in the prior art.

Laboratory Characterization of the uni-sourced LBDDs.

For further development of the products of the current invention, there is a need for characterization of each, and every emulsion type. According to the current state of the art critical quality attributes of nanoemulsions include droplet size, polydispersity index, zeta potential, and drug loading capacity. Droplet Size and Surface Charge (Zeta Potential). The droplet size distribution of microemulsion vesicles can be determined by either electron microscopy or light scattering technique. The physical stability of the emulsions was evaluated by static multiple light scattering using a vertical scan analyzer. Factors related to product physical stability are particle size distribution, the type of surfactant, and the antioxidant activity related to vitamins C and E, and fatty acids. Rheological Characterization of the Emulsions, i.e., the flow behavior of the emulsions was evaluated in a controlled shear rate rheometer. Transmission electron microscopy was used to investigate the shape and surface morphology of the nanoemulsion and droplets in aqueous dispersions. ((Mazonde et al., 2020; Karen Fuentes et al., 2021; Shrestha et al., et al., 2014).

Lipid crystallinity of nanoemulsions is measured using a thermo-analytical technique; nuclear magnetic resonance (NMR)-based techniques have also been used to study the types, structure, and diffusion properties of components in nanoemulsions; and electron microscopy (EM) techniques is used for the visualization of the microstructure of nanoemulsions (Aswathanarayan and Vittal, 2019). In addition, atomic force microscopy is capable of measuring interfacial interaction forces of nanoemulsion droplets was also reported for its ability to differentiate the nanoemulsion properties prepared from different surfactant types (Thao Minh Ho, et al., 2021).

Indications for use of fermented uni-sourced Nigella nanocomposition of this invention in mammals including humans.

The pharmacological activity and indications for use of Nigella sativa nano-formulation of this invention can be divided into cosmetic and therapeutic, local, and systemic indications, including a unique oral formulation to relax coily hair from the roots.

In other embodiment, the composition of the current invention is used in combination with conventional treatment of the said illness, by combining Nigella sativa uni-sourced or fermented uni-sourced nanocompositions with the latter by any method known in the prior art.

### Cosmetic Indications Include:

A unique indication for the use of fermented uni-sourced Nigella LBDDs is to relax the coily, kinky and wavey hair shaft by relaxing the smooth muscle of the hair follicles known as “arrector pili” muscle. We hypothesize that a strong and continuous contraction of arrector pili is the principal cause underlying the coily, kinky and wavey shape of the hair shaft. An example of Nigella sativa components that have smooth muscle relaxing effect are alpha-hederin and carvacrol. The antispasmodic effect of dithymoquinone and TQ was also demonstrated (Mukhtar et al., 2019). The smooth muscle relaxing action of other bioactives of Nigella sativa need to be investigated.

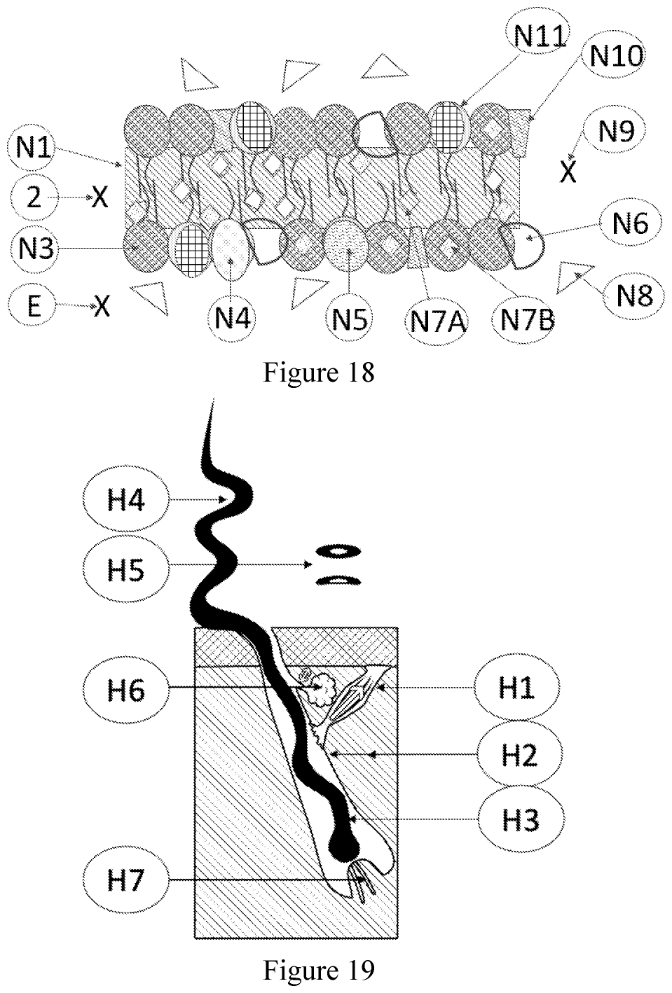

FIG. 19 depicts the anatomical basis of the coily/kinky hypothesis that form the basis of relaxing (straightening) coily, kinky and curly hair by the oral formulation of this invention, wherein a strong and contracted arrector pili smooth muscle (H1) of the hair follicle is located within the skin dermis tissue, being attached to the hair root sheet (H2) and is pulling on it is resulting in coiled hair shaft (H3) lying within the hair follicle and on the external part of the hair shaft (H4), this pulling effect of the arrector pili will cause a flat section of the hair shaft (H5). In addition, the contracted arrector pili will squeeze the sebaceous oil glands of the hair root (H6) with kinking of the sebaceous ducts causing dry hair and will pull on the blood vessels of the hair root (H7) resulting in thinning of the shaft.

The products of the current invention will be the first-in-class oral dosage forms for relaxing coily, kinky and wavy hair types. The mechanism of action is to relax the arrector pili, and hence release the pulling action on the hair follicle and the hair shaft, i.e., relaxing the hair. It also helps the production and release the sebum oil treating the dryness and fizziness, improve the blood supply to improve the hair health, and eventually changing the cross section to a more rounded shape.

The uni-sourced LBDDs and the fermented LBDDs from Nigella sativa are indicated for local application and systemic administration to treat alopecia areata.

The products are indicated as antiaging therapy for local and systemic administration as they have antioxidant and anti-apoptosis activity, reduce skin redness, nourishes the skin and aids in healing process, and lighten the skin color. In the cosmetic field Nigella sativa have been used by Cleopatra and Nefertiti in ancient Egypt!

### Medical Indications Include:

The uni-sourced LBDDs and the fermented LBDDS are indicated for use in the treatment of human diseases based on Nigella sativa pharmacologic activities:

(A) Treatment of metabolic syndrome with obesity, dyslipidemia and/or diabetes and its complications, and hypertension.

(B) Anticancer treatment with one or more group of functions including anti-proliferative, anti-angiogenesis, pro-apoptotic, anti-oxidant, cytotoxic, anti-mutagenic, and anti-metastatic effects, used as an add-on medication or in combination with conventional anti-cancer drugs

(C) Anti-COVID-19 to treat COVID-19 as an outpatient drug to take home or for inpatients as add-on treatment formulated in an oral dosage form that enhance innate immune response to the SARS-CoV-2 virus

(D) Respiratory diseases as an add-on treatment of tuberculosis. Mainly alpha-hederin and TQ are hepatoprotective against current anti-tuberculosis therapy or in asthma and allergic rhinitis, and chronic obstructive pulmonary disease.

(E) Anti-viral effect: in the treatment of HIV, hepatitis C and B.

(F) Neuroprotective in central nervous system diseases including seizure, multiple sclerosis, Parkinson, Alzheimer's disease, and memory enhancement.

(G) Anti-bacterial activity: mainly against antibiotic-resistant bacteria.

(H) Immunomodulation/enhancement of innate immune responses.

(I) Anti-inflammatory and analgesic effect: in the treatment of Crohn's disease, facial palsy, inflammatory arthritis, degenerative osteoarthritis, and recurrent cystitis.

(J) Antioxidant effect: is effective in the prevention of chronic and degenerative diseases associated with oxidative stress that is useful as anti-aging treatment, and the treatment of rheumatoid arthritis, diabetes mellitus, and hepatic injury.

(K) Treatment of infertility and reproductive system diseases.

(L) The products of this invention can be used as a dietary supplement.

### Example 2: Nanoemulsification of Cannabaceae Family to Produce Fermented Uni-Sourced Cannabinoid-Loaded Nanocomposition

Background of Cannabis family.

A second example of an oil-producing medicinal plant related to this invention is the genus Cannabis, family Cannabaceae, includes marijuana, Cannabis sativa (hemp) and Cannabis indica, weed, pot and others. The genus cannabis is a rich source of cannabinoids, that are the bioactive phytochemicals mainly cannabidiol (CBD), and delta-9-tetrahydrocannabinol (THC).

The word marijuana refers to parts of or products from the plant Cannabis that contains substantial amount of THC. THC is the substance that's primarily responsible for the effect of being high.

Cannabis plants are known for their recreational, nutraceutical, cosmetic, and medicinal uses

The composition of marijuana and hemp.

The main bioactive phytochemicals of Cannabis genus are delta-9-tetrahydrocannabinol (THC) and cannabidiol (CBD), Oil blend contains minor types of cannabinoids, including cannabigerol (CBG) and cannabinol (CBN), CBD1, CB2, cannabichromenic acid, and others. Some cannabis plants contain very little THC, while oils for medical use contain mostly CBD. The cannabinoids are highly hydrophobic and TSH contents are lost through exposure to light and unstable moisture conditions.

The plant's raw total oil extracted from dried or fresh cannabis is naturally thick and viscous. A supercritical CO2 extraction of the whole plant method produces a dark green oil. The raw oil includes the total oil and the volatile oil.

The volatile oil contains the bioactive phytochemicals cannabinoids and terpenes.

The total oil is composed of polyunsaturated fatty acids, the most abundant are linolenic acid, linoleic acid, palmitic acid, oleic acid, and saturated fatty acids mainly stearic and palmitic acid. (Piovesana et al., 2021; Gulluni et al., 2018;). The oil also contains small amount of phospholipids, phytosterols, and vitamin E (Hazekamp et al., 2010).

The non-cannabinoids like terpenes, flavonoids, alkaloids, phenols, proteins, and high carbohydrate and protein contents, cannabispiron and its oxidative derivative terpenoid fraction (Pavlovic et al., 2019; Siano et al., 2019; Bakain et al., 2020; Radwan et al., 2021; Gul(luni et al, 2018; Piovesana et al., 2021;). Terpenes, mono/di/tri terpenes, and terpenoid-like substances are non-psychoactive compounds that are responsible for the scent and taste of distinct cannabis. Other components are Antioxidants, saponins and flavonoids like limonene. It is not clear whether alpha-hederin is a constituent of marijuana, but there is reference to saponins, including a triterpene saponin as a natural product that is found in herbs, including marijuana, in the treatment of pain (Guimaraes et al., 2013).

Manufactured THC is also available: Delta-8 THC are typically manufactured from CBD.

Current methods of cannabis oil extraction.

Steam distillation is the most efficient form of Cannabis oil production. Only expressed oils that have not come in contact with heat or aerial oxidation may meet the conditions of a true plant essential oil. Solvent-based extraction of medical marijuana include Soxhlet, ultrasonic-assisted, microwave-assisted extraction that requires a solvent to complete the extraction process. a variety of solvents can be used to extract cannabis oil and its cannabinoids including ethanol, butane, propane, hexane, ether, methanol, acetone, and olive oil (Lazarjani et al., 2021; Morenos et al., 2020).

The plant parts can be heated to low temperature to produce decarboxylated active THC and CBD, although cannabis tincture can be prepared by cold alcohol extraction method.

When the extracted sample is collected, the solvent should be evaporated because some extraction solvents may be toxic if not properly purged from the marijuana oil, residual solvents affect the taste of the product. In addition, the solvent extraction extracts chlorophyl that affect the color and quality of the cannabis oil and need to be washed.

The other method is oil infusion (tincture) which is a straightforward method, by boiling the plant material with vegetable oil, e.g., olive or coconut oil, for a couple of hours.

Oil extraction using propane and ethanol are one of the most efficient methods that strip the unwanted material from the desired chemicals. The vape is mostly a water-soluble form of CBD infused with terpenes.

Cannabis companies are learning new ways to manipulate terpenes to improve flavor and give a different kind of highs. Extracting terpenes can give a superior synergistic taste and smell (https.//mashable.com/article/weed-cannabis-terpenes).

Current cannabis nanoemulsion production.

Recently, efforts to produce new improved nanoemulsion for oral delivery application characterized by enhanced stability (Fathordoobady et al., 2021). A nanoemulsion that enhance the water compatibility and bioavailability of cannabinoids has been developed by Nanogen Labs Inc. (patent pending).

A nanoemulsion of hempseed oil derived from Cannabis sativa L. was prepared for oral delivery application prepared from hempseed oil using lecithin as a surfactant in two methods microfluidization and ultrasonication containing non-psychoactive cannabinoid compounds CBDA and CBD. These compounds are biologically active and demonstrate anti-convulsive, anti-epileptic and antimicrobial effects (Fathordoobady et al., 2021).

Cannabis beverage is being developed as oil-in-water emulsion with the general formula of cannabis oil (5%)+emulsifier (5%)+carrier oil+antioxidant (1%). The cannabis oil is extracted using supercritical CO2 technique. Emulsifiers include Quilija saponin, lecithin, lysolecithin, xanthan gum, pectin, gum Arabic, whey protein, and caseinates. Carrier oils include walnut oil, avocado oil, grapeseed oil, coconut oi, sesame oil, sunflower oil, soybean oil, sweet almond oil. The emulsion is further homogenized to adjust the particle size with a choice of producing either opaque (150 nm), translucent (100-50 nm), or transparent (10 nm) products that are a liquid delivery system for the bioactive phytochemicals. https.//leherbe.com/images/pdf/the_art_and_science_of_cannabis_beverages.pdf

Current cannabis fermented products.

Recently, companies are producing fermented CBD-alternative as an alternative to traditional production for innovative and improved products, e.g., production of bio-CBG without the need for Cannabis plant (Creoingredients.com).

Current indications for use of Cannabis.

According to the current state of the art, the indication for use of THS and CBD are recreational, medicinal, nutraceutical, cosmetic, health and wellness, and antiaging (Bhaskar et al., 2021; Al Ubeed et al., 2022; Lazarjani et al., 2021; Gulluni et al., 2018; www.medicalmarijuanainc.com).

Wherein, medical marijuana refers to using the whole Cannabis plant, or the plant's oil or basic extracts that either are CBD dominant with minimal THC mostly derived from hemp or might contain THC and will cause the patient to be high when taking the medicine.

Novel Fermented Uni-sourced Cannabis Nanoemulsion

The extraction and nano-emulsification of Cannabis oil producing plant is a second example of this invention, wherein the novel products includes uni-sourced cannabinoid-loaded nanoemulsion, and a fermented uni-sourced cannabinoid-loaded nanoemulsion, wherein Cannabis plant act as a single source for all the components of the nanoemulsion providing the cannabinoid family of bioactive phytochemicals, cannabis total and volatile oil, fatty acids, phospholipids, saponins, terpenes, carbohydrates, proteins, and minerals.

Herein, the term nanoemulsion is referring to the group of lipid-based drug-loaded delivery system (LBDDs), including nanoemulsion, microemulsion, micelle, liposomes, and others.

Herein the extraction and emulsification process are using organic acids as a solvent, and in a preferred embodiment the solvent is ethanoic acid.

In this novel 4E process the cannabis oil is being extracted from its intracellular location, droplet by droplet, using ethanoic acid, and then spontaneously emulsified in the aqueous phase that contains cannabis surfactants and saponin.

The process and the products of novel Cannabis nanoemulsification is similar to the above-described invention in many aspects, selected from the figures are the following:

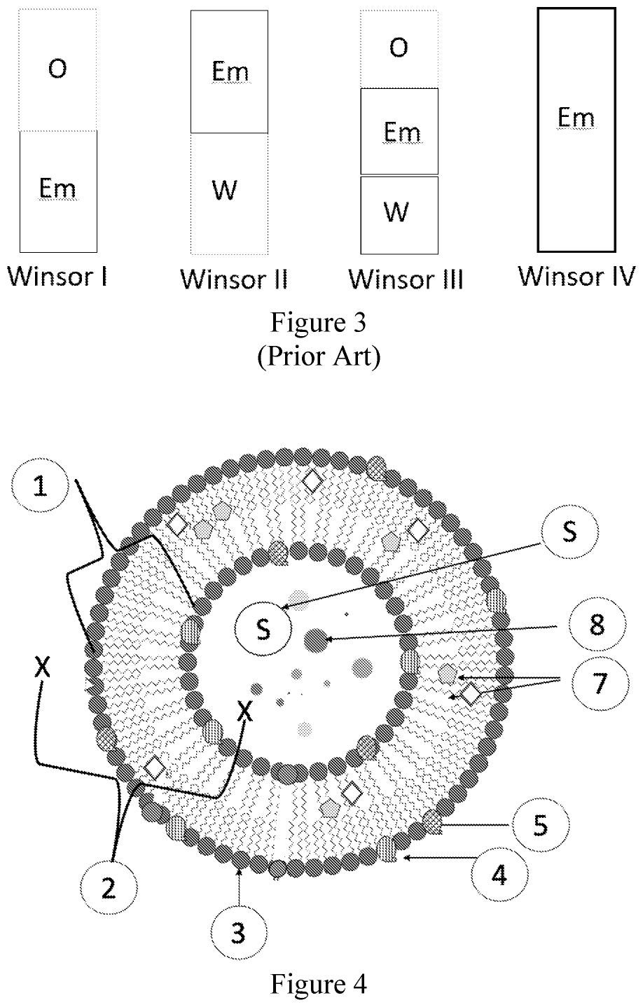

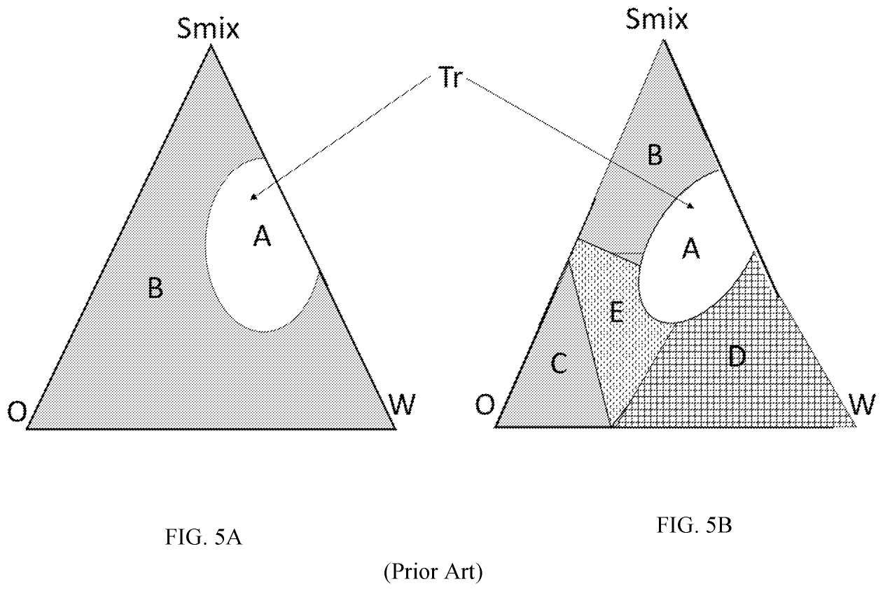

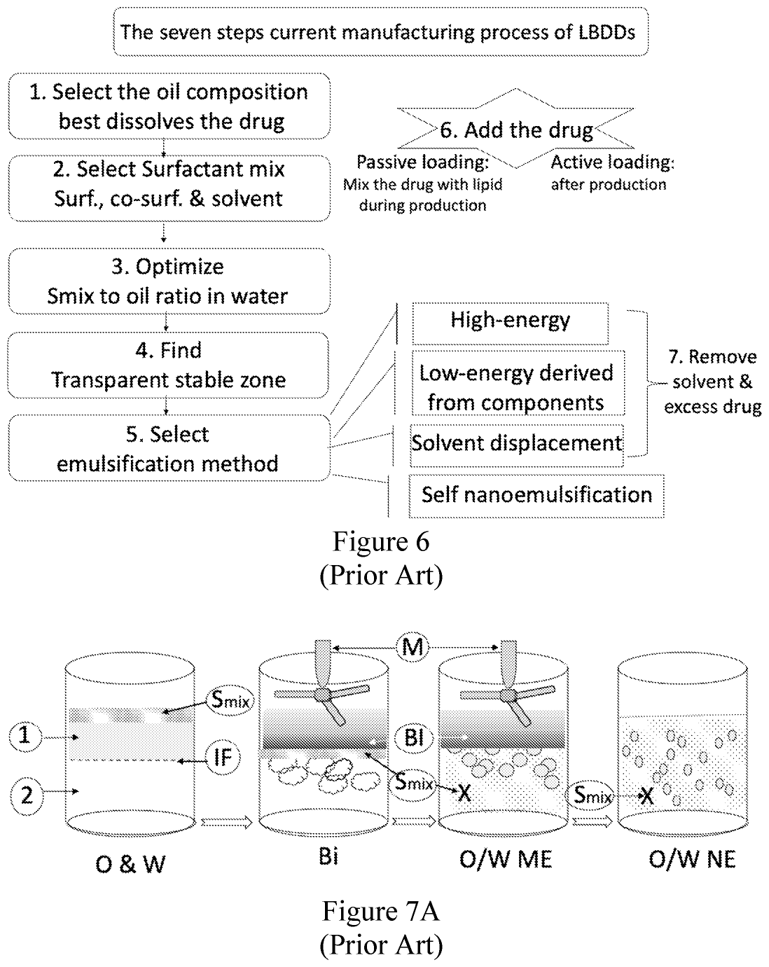

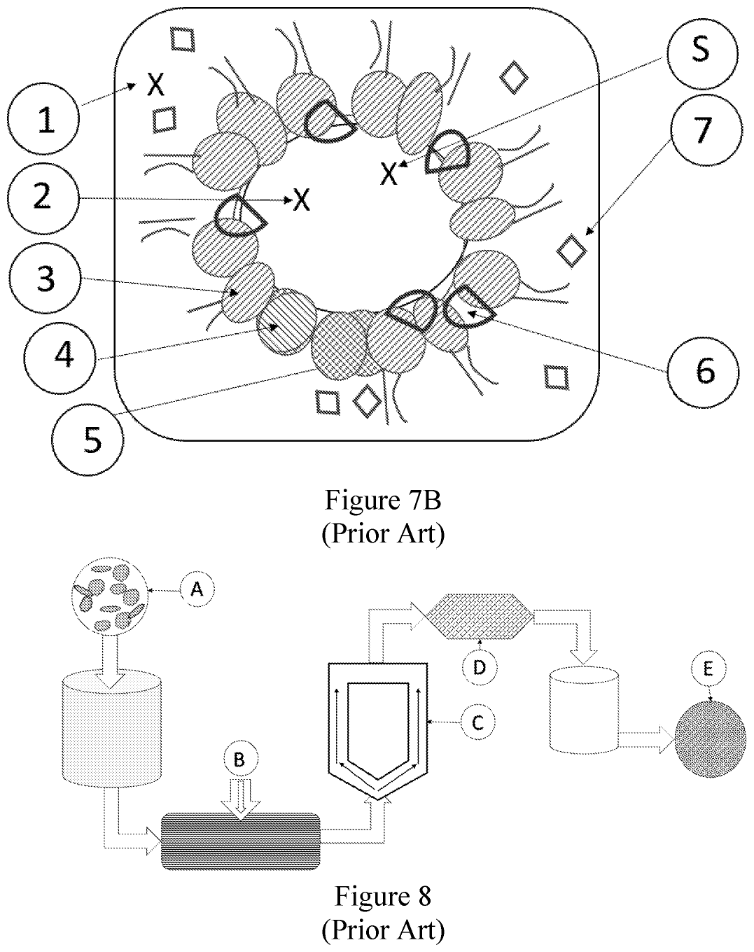

FIG. 3 depicting the physical characteristics of nanoemulsions.

FIG. 9 depicting the concept of the homogenous uni-sourced lipid-based drug delivery system carrying the bioactive phytochemicals (CBD and/or THC).

FIG. 11 depicting the stepwise process of nanoemulsification of the current invention, wherein the aqueous ethanoic acid dissolves the cellulose of the cell membranes and extract the intracellular oil and lipids including Cannabis volatile oil (C1) that will form the core of the nanoparticle, Cannabis phospholipids (C3); and the saturated and unsaturated fatty acids (C4).

FIG. 12 that describe the biofermentation and esterification of the 4E nanoemulsification process.

FIG. 13 depicting the timeline of the 4E process (extraction, emulsification, ethyl alcohol fermentation, and esterification also applies to Cannabis nanoemulsification.

FIG. 14 depicting the clock-wheel optimization also applies to Cannabis nanoemulsification.

Which part of Cannabis/hemp are used in this extraction process?

Herein, the oil plant that is selected as a raw material source of the nanoformulation is from the family Cannabaceae, include marijuana, Cannabis sativa (hemp) and Cannabis indica, weed, pot that are rich in cannabinoids mainly CBD and THC.

The parts of the plant used can be the flowering buds, leaves, seeds, or the whole plant known in the art. The hemp extraction may use any part of the plant, mainly the flowering buds and complete inflorescence flower head.

What are the components of the uni-sourced Cannabis LBDDs?

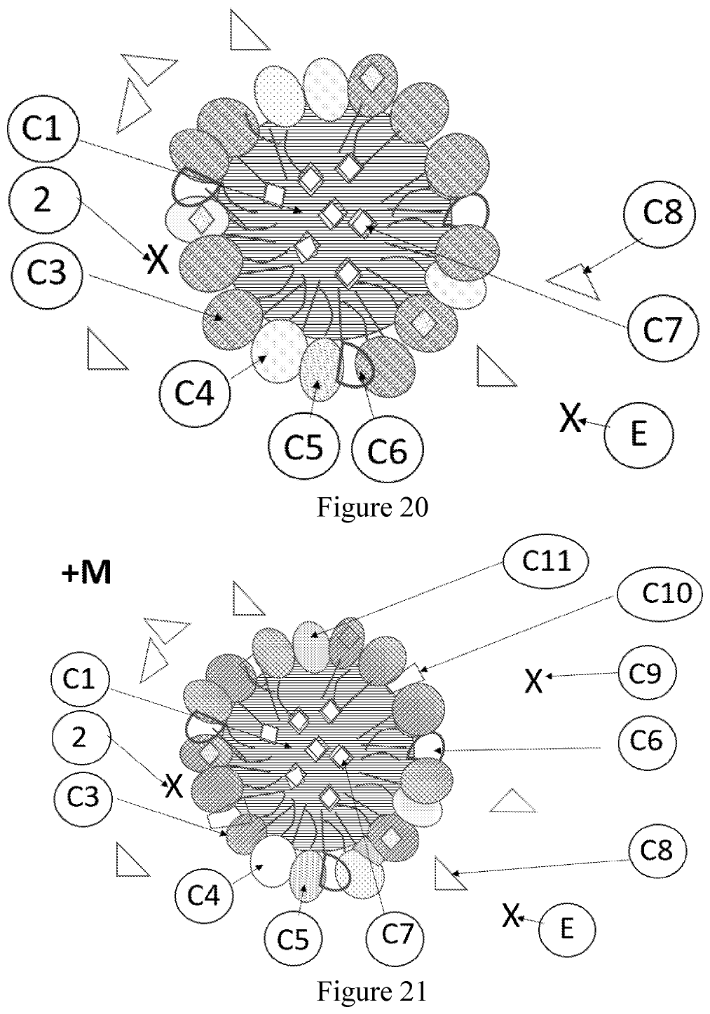

FIG. 20 demonstrates an example of the components of Cannabis nanoemulsion and their molecular assembly, wherein the uni-sourced nanoemulsion/microemulsion is consisting of nanoparticle (nanodroplet) assembled as a core and a shell, wherein the core is composed of Cannabis volatile oil that carry the lipophilic CBD and THC cannabiboids; wherein the nanoemulsion is composed of an oil phase (C1) and a water phase (2) containing aqueous ethanoic acid solvent (E). The letter (C) indicate that the components are derived from the herb Cannabis, Wherein the core represents the oil phase (C1) of the particle which is composed of Cannabis volatile oil; wherein the shell is composed from other components derived from Cannabis oil including phospholipid (C3), saturated and unsaturated fatty acids (C4). The shell also composed of Cannabis proteins (C5), and Cannabis saponins (C6) to produce a tightly assembled shell that carry, protect, and deliver the lipophilic unstable bioactives. The lipophilic Cannabis bioactive cannabinoids are preloaded in the volatile oil core mostly CBD and THC (C7) and the hydrophilic bioactive cannabinoids mostly saponins and terpenes (C8) are dissolved in the surrounding water phase.

What are the components of the fermented uni-sourced Cannabis LBDDs?

FIG. 21 demonstrates an example of the components of the fermented uni-sourced drug loaded nanoemulsion and their molecular assembly, wherein the uni-sourced nanoemulsion is consisting of nanoparticle (nanodroplet) assembled as a core and shell and is composed of the components C1-C8; wherein the product is further fermented by the addition of mother of vinegars (M); wherein the fermentation process of uni-sourced Cannabis LBDDS will convert the carbohydrate components in an acidic medium and result in the production of Ethyl alcohol (C9); wherein further esterification process of uni-sourced Cannabis LBDDS will result in the production of Ethyl esters (C10) and esterified fatty acids (C11) incorporated in the shell resulting in tight molecular assembly for improved carrier function.

The formation of “mother of Cannabis” plant raw material.

In one embodiment, the incubation of the nanoemulsion in the presence of the mother of vinegar will result in the formation of the “mother of Cannabis”, which is a thick gelatinous opaque yellowish biofilm disc formed on the surface of the liquid composition composed of cellulose and acetic acid bacteria that feeds on fermentation products. It grows in size and form multiple layers. It can be formed under both anaerobic and during aerobic incubation. The mother of vinegar is formed when we use the ethanoic acid solvent that is unpasteurized and contains the mother of vinegar, and the cellulose is produced by the action of ethanoic acid dissolving the cellulose contents of the cell wall of Cannabis while extracting the seed oil.

In one preferred embodiment, the differences in the solvent concentration and the Cannabis flowers or buds grinding size will result in a milky opaque granular composition, suggestive of a liposomal type LBDDs; where the liposome is a vesicle with a shell composed of phospholipid bilayer incorporating the oil phase; the bilayer is enclosing a water droplet core and dispersed in an aqueous phase.

In other embodiment, the differences in the solvent concentration and the Cannabis flowers or buds grinding size will result in the formation of other types of lipid-based cannabinoid-loaded drug delivery system (LBDDs) like micelle and white crystals.

In other embodiment the raw hemp oil can be used as the raw material for the production of LBDDs of this invention.

In yet another embodiment a combination of synthetic cannabinoids with the cannabis raw material rich in cannabinoid family of bioactive phytochemicals are mixed to enrich the final product.

What are the products included under Cannabis LBDDs?

ALL the above-described LBDDS are included under one term “fermented uni-sourced cannabinoid-loaded LBDDS” in drafting the claims. This term includes one or more of the said Cannabis nanoemulsion, macroemulsion, liposomes, micelles, crystals, and mother of Cannabis that can carry, protect, and deliver cannabinoids including CBD or THS.

Formulation of Cannabis products of this invention might need the addition of coloring, flavoring, PH adjusting, fillers, stabilizers, excipients or any other additives known in the prior art.

The products that can be manufactured from fermented unisourced Cannabis-loaded LBDDS.

In one embodiment it can be used as Cannabis infused beverage.

In other embodiment, the liquid extract can be concentrated in the form of a tincture.

In other embodiment, the fermented uni-sourced Cannabinoid-loaded LBDDs can be incorporated in edibles ((brownies, gels, gummies, and supplements), formulated as capsules, tablets, smokable products.

CBD a variety of form of supplements for overall health and wellness.

The product can be presented as a smoke or vape of marijuana or CBD.

THC can also come in different forms including oils, tinctures, capsules, edibles, and smokable products.

Dosage of fermented unisourced Cannabis-loaded LBDDS.

The dosage described herein is guided by the current products available for consumers, and the dosage is not a limiting factor of the invention.

Calculation of CBD or HTS per unit volume of the product is calculated as: the weight of the Cannabis raw material X CBD or TSH content in 1 gm of the raw material divided by the total volume of the composition produced. And accordingly, the single dose in cubic centimeters/dose/day is calculated. Any standard methods for the calculation can be used.

Legal hemp must contain 0.3% THC or less.

Recreational Cannabis contains CBD formulated as gummies might contain 10-25 mg per piece of CBD for a once daily dosage.

When a higher dose is required CBD gummies contains 50 mg CBD with 2 mg THC, for a dose of 1 gummy daily or as prescribed.

Medical marijuana recommended dosage is between 5-40 mg CBD twice daily, and clinicians may consider adding THC at 2.5 mg up to 40 mg/day.

Indication for use of fermented unisourced Cannabis-loaded LBDDS.

Cannabis plants are known for their recreational, medicinal, nutraceutical, and cosmetic uses. Medical marijuana is characterized by psychoactive, narcotic, as well as medicinal actions used for the treatment of various ailments or conditions, to reduce pain and inflammation, multiple sclerosis, anti-epilepsy, anxiolytic, antipsychotic, to treat sleep disorders, glaucoma, vascular disorders, autism, schizophrenia, and to prevent Alzheimer's disease and Parkinson.

The general properties of cannabinoids include antidepressant, relaxant, anxiolytic, sedative, antimicrobial, and antioxidants.

Cosmetic Cannabis products are indicated for local application that is prepared by mixing CBD oil with hempseed oil used in skincare, acne, sensitive skin rashes, eczema, and psoriasis, sleep, health, and overall wellness. Hempseed oil can be mixed with a carrier oil and is used for muscle soreness, skin blisters or chafing.

Advantages of the 4E Cannabis nanoemulsification process.
the advantages of the invention extraction process are described in pages 34-35 above

### Example 3: Nanoemulsification of Curcuma Species to Produce Fermented Uni-sourced curcuminoids-loaded Nanocomposition

### Background of Curcuma Species

A third example of an oil-producing medicinal plant related to this invention is the Curcuma species including Curcuma longa which is an oil-producing plants with pharmaceutical interest and clinical benefits.

The curcuma composition was reviewed by (Dosoky and Setzer, 2018), and Curcumin nanoemulsion was reviewed by (Adena et al., 2021).

In the current invention:

Composition of Curcuma longa uni-sourced curcuminoid-loaded LBDDs nanoemulsion

Many of the biological activities of Curcuma species can be attributed to nonvolatile curcuminoids, volatile chemicals. Essential oils, terpenoids, flavonoids, phenypropanoids, Beta-pinene and sesquiterpenes.

### A. Biological Activities of Curcuma L Plant and its Oil

Members of the genus Curcuma L. have been used in traditional medicine for centuries for treating gastrointestinal disorders, pain, inflammatory conditions, wounds, and for cancer prevention and antiaging, among others. in general, have shown numerous beneficial effects for health maintenance and treatment of diseases. Essential oils from Curcuma spp., particularly C. longa, have demonstrated various health-related biological activities and several essential oil companies have recently marketed Curcuma oils.

Curcuma L. are known for containing terpenoids, flavonoids, phenypropanoids and sesquiterpenes, which have antitumor activities. Some Curcuma essential oils have remarkable antioxidant and antimicrobial activities that make them ideal candidates for use in pharmaceutical and cosmetic industries. possess central nervous system depressant, analgesic, antioxidant, anti-inflammatory, antiplatelet, cytotoxic, hypotriglyceridemic, antibacterial, and antifungal activities. Curcuma amada rhizome essential oil and ethanolic extracts showed hepatoprotective effects against carbon tetrachloride-induced hepatotoxicity in male Wister rats mainly due to their strong antioxidant activities

U.S. provisional application No. 61/163,688, March 2009, and European patent #EP2410874A1 titled” Nutritional composition comprising curcuminoids and method of manufacture” providing a composition with selected ratio of curcuminoids having improved biological activity, bioavailability, and reduced color impact, by solubilizing the curcuminoids in a polar oil in an aqueous emulsion.

In this invention, the above-described process and product innovative product applies to the manufacturing of the novel uni-sourced curcuminoid-loaded LBDDs nanocomposition, and the novel fermented uni-sourced curcuminoid-loaded LBDDs nanocomposition.

When used in this specification and claims, the terms “comprises” and “comprising” and variations thereof mean that the specified features, steps, or integers are included. The terms are not to be interpreted to exclude the presence of other features, steps or components.

The invention may also broadly consist in the parts, elements, steps, examples and/or features referred to or indicated in the specification individually or collectively in any and all combinations of two or more said parts, elements, steps, examples and/or features. In particular, one or more features in any of the embodiments described herein may be combined with one or more features from any other embodiment(s) described herein.

Protection may be sought for any features disclosed in any one or more published documents referenced herein in combination with the present disclosure. Although certain example embodiments of the invention have been described, the scope of the appended claims is not intended to be limited solely to these embodiments. The claims are to be construed literally, purposively, and/or to encompass equivalents.

1. A uni-sourced Nigella-loaded lipid-based drug delivery system comprising:
an oil phase comprising a Nigella sativa volatile oil,
wherein the oil phase is loaded with thymoquinone;
a water phase,
wherein the water phase contains alpha-hederin;
a surfactant selected from the group consisting of phospholipids, saturated fatty acids, unsaturated fatty acids, cholesterol, alpha-hederin, and protein; and
a solvent comprising aqueous ethanoic acid,
wherein the oil phase, the surfactant, and the solvent are sourced from a single species of a plant.

2. The lipid-based drug delivery system of claim 1:
wherein the lipid-based drug delivery system is incubated with mother of vinegar;
wherein the oil phase further comprises at least one additional Nigella sativa volatile oil;
wherein the water phase at least one additional component selected from the group consisting of a water-soluble bioactive phytochemical, a protein, a mineral, and a carbohydrate; and
wherein the aqueous ethanoic acid has a concentration of 0.01-50%.

3. The lipid-based drug delivery system of claim 1, further comprising:
mother of vinegar, which ferments with Nigella sativa carbohydrates to generate ethyl alcohol; ethyl ester and fatty acid esters esterified from at least a portion of the ethyl alcohol, forming an emulsifier and a stabilizer;
mother of Nigella sativa, formed from the fermentation process; and
a white precipitate formed from the fermentation process.

4. The lipid-based drug delivery system of claim 2, further comprising:
mother of vinegar, which ferments with Nigella sativa carbohydrates to generate ethyl alcohol;
ethyl ester and fatty acid esters esterified from at least a portion of the ethyl alcohol, forming an emulsifier and a stabilizer;
mother of Nigella sativa, formed from the fermentation process; and
a white precipitate formed from the fermentation process.

5. The lipid-based drug delivery system of claim 1:
wherein the lipid-based drug delivery system is administered to a mammal in a dosage calculated based on a content of bioactive phytochemicals in the lipid-based drug delivery system; and
wherein the lipid-based drug delivery system is in a form selected from the group consisting of a liquid, a solid, an intranasal drop, a spray for inhalation, an intravenous solution, and a topical solution.

6. The lipid-based drug delivery system of claim 5, wherein the lipid-based drug delivery system is formulated for an oral dosage for a cosmetic indication used to straighten coily, kinky, or curly hair, lighten skin color, or provide antiaging therapy.

7. The lipid-based drug delivery system of claim 5, wherein the lipid-based drug delivery system is formulated for an oral dosage administered to treat metabolic syndrome with obesity, dyslipidemia and/or diabetes and its complications, and hypertension.

8. The lipid-based drug delivery system of claim 5, wherein the lipid-based drug delivery system is formulated for an oral dosage administered to treat cancer with one or more group of functions including anti-proliferative, anti-angiogenesis, pro-apoptotic, anti-oxidant, cytotoxic, anti-mutagenic, and anti-metastatic effects, used as an add-on medication or in combination with conventional anti-cancer drugs.

9. The lipid-based drug delivery system of claim 5, wherein the lipid-based drug delivery system is formulated for an oral dosage administered to treat COVID-19 as an outpatient drug to take home or for inpatients as add-on treatment formulated in an oral dosage form that enhance innate immune response to the SARS-CoV-2 virus.

10. The lipid-based drug delivery system of claim 5, wherein the lipid-based drug delivery system is formulated for an oral dosage administered to treat respiratory diseases as an add-on treatment of tuberculosis.

11. The lipid-based drug delivery system of claim 5, wherein the lipid-based drug delivery system is formulated for an oral dosage administered to treat viral diseases including the treatment of HIV, hepatitis C, and hepatitis B.

12. The lipid-based drug delivery system of claim 5, wherein the lipid-based drug delivery system is formulated for an oral dosage administered to treat Crohn's disease, facial palsy, inflammatory arthritis, degenerative osteoarthritis, and recurrent cystitis or locally to treat alopecia areata.

13. The lipid-based drug delivery system of claim 5, wherein the lipid-based drug delivery system is formulated for an oral dosage administered to treat rheumatoid arthritis, diabetes mellitus, and hepatic injury.

14. A method of producing a uni-sourced lipid-based drug delivery system comprising:
grinding parts from at least one plant selected from the group consisting of Nigella sativa, Cannabis sativa, and Curcuma longa;
adding an aqueous solution of ethanoic acid to the plant parts to extract components, wherein the components comprise lipophilic components, fixed oil components, surfactants dissolved in a water phase, wherein the surfactants stabilize the components; and
allowing the components to emulsify.

15. A uni-sourced Cannabinoid-loaded lipid-based drug delivery system comprising an oil phase and water phase with a surfactant system and a solvent, wherein:
the oil phase comprises a Cannabis volatile oil;
the oil phase is loaded with Cannabis bioactive phytochemical comprising at least one CBD, THC, or other cannabinoids;
the water phase contains a Cannabis hydrophilic bioactive compound comprising of at least saponin, terpenes, flavonoids, and minerals;
the surfactant system is derived from the Cannabis comprising at least one surfactant chosen from the group consisting of phospholipids, saturated and unsaturated fatty acids, saponin, and protein;
wherein the solvent is aqueous ethanoic acid with dual action of oil extraction from its intracellular location and oil emulsification.

16. A fermented uni-sourced Cannabinoid-loaded lipid-based drug delivery system comprising an oil phase and water phase with a surfactant system and a solvent, wherein:
the oil phase comprises Cannabis volatile oil comprising at least one CBD, THS, or other cannabinoids;
the water phase contains Cannabis hydrophilic bioactive phytochemicals comprising of at least saponin, terpenes, flavonoids proteins, and minerals;
the surfactant system is derived from Cannabis comprising at least one surfactant chosen from phospholipids, saturated and unsaturated fatty acids, saponin, and protein;
the solvent comprises aqueous ethanoic acid with the mother of vinegar that ferments Cannabis carbohydrates to generate ethyl alcohol that is further esterified into ethyl ester and fatty acid esters that act as additional emulsifiers and stabilizers; and
wherein mother of Cannabis is produced through fermentation.

17. The fermented uni-sourced cannabinoid-loaded lipid-based drug delivery system of claim 16, wherein the fermentation process produces a white precipitate.

18. A method of use of the cannabinoid-loaded lipid-based drug delivery system of claim 15, wherein the lipid-based drug delivery system is formulated for administration to a mammal in need for such treatment, wherein:
a dosage is calculated according to the contents of bioactive phytochemicals in the formula;
a daily recommended dosage is administered as a single dose or divided into multiple doses;
wherein a duration of treatment is selected from one day therapy or longer duration according to the consumer needs;
wherein the formulation is produces include at least one formulation chosen from the group consisting of a liquid oral dosage form of beverages, tincture, edibles, capsules, tablets, vape, dietary supplement, and local skin medication.

19. The method of claim 18, wherein the Cannabinoid-loaded LBDDS is used to treat a consumer for indication chosen from the group consisting of recreational, wellness and overall health, or medicinal purposes.

20. A fermented uni-sourced curcuminoid-loaded LBDDs comprising an oil phase and water phase with a surfactant system and a solvent, wherein:
the oil phase comprises Curcuma volatile oil;
the water phase contains Curcuma hydrophilic bioactive phytochemicals comprising of at least saponin, terpenes, flavonoids proteins, and minerals;
the surfactants system derived from Cannabis comprising at least one surfactant chosen from phospholipids, saturated and unsaturated fatty acids, terpenoids, beta-pinene, protein, and other components;
the nanocomposition is formulated for oral dosage form to be used for one or more of nutraceutical, overall health and lifestyle, medicinal, and cosmetic indications.
